SEQUENCE LISTING (77 sequences)

SEQ ID NO: 1 - DNA, 21 bp, synthetic construct
  misc_feature 1..19: note = RNA
  misc_feature 20..21: note = DNA
  source 1..21: mol_type = other DNA
agatggagagccttgtccctt

SEQ ID NO: 2 - DNA, 21 bp, synthetic construct
  misc_feature 1..19: note = RNA
  misc_feature 20..21: note = DNA
  source 1..21: mol_type = other DNA
gggacaaggctctccatcttt

SEQ ID NO: 3 - DNA, 21 bp, synthetic construct
  misc_feature 1..19: note = RNA
  misc_feature 20..21: note = DNA
  source 1..21: mol_type = other DNA
cgtggtaagagaattcctttt

SEQ ID NO: 4 - DNA, 21 bp, synthetic construct
  misc_feature 1..19: note = RNA
  misc_feature 20..21: note = DNA
  source 1..21: mol_type = other DNA
aaggaattctcttaccacgtt

SEQ ID NO: 5 - DNA, 21 bp, synthetic construct
  misc_feature 1..19: note = RNA
  misc_feature 20..21: note = DNA
  source 1..21: mol_type = other DNA
gagaaaagctgtctttatttt

SEQ ID NO: 6 - DNA, 21 bp, synthetic construct
  misc_feature 1..19: note = RNA
  misc_feature 20..21: note = DNA
  source 1..21: mol_type = other DNA
aataaagacagcttttctctt

SEQ ID NO: 7 - DNA, 21 bp, synthetic construct
  misc_feature 1..19: note = RNA
  misc_feature 20..21: note = DNA
  source 1..21: mol_type = other DNA
cttactaaaggacctcatgtt

SEQ ID NO: 8 - DNA, 21 bp, synthetic construct
  misc_feature 1..19: note = RNA
  misc_feature 20..21: note = DNA
  source 1..21: mol_type = other DNA
catgaggtcctttagtaagtt

SEQ ID NO: 9 - DNA, 21 bp, synthetic construct
  misc_feature 1..19: note = RNA
  misc_feature 20..21: note = DNA
  source 1..21: mol_type = other DNA
attatcaaaacaatgtttttt

SEQ ID NO: 10 - DNA, 21 bp, synthetic construct
  misc_feature 1..19: note = RNA
  misc_feature 20..21: note = DNA
  source 1..21: mol_type = other DNA
aaaacattgttttgataattt

SEQ ID NO: 11 - DNA, 21 bp, synthetic construct
  misc_feature 1..19: note = RNA
  misc_feature 20..21: note = DNA
  source 1..21: mol_type = other DNA
atgtctgaagcaaaatgtttt

SEQ ID NO: 12 - DNA, 21 bp, synthetic construct
  misc_feature 1..19: note = RNA
  misc_feature 20..21: note = DNA
  source 1..21: mol_type = other DNA
aacattttgcttcagacattt

SEQ ID NO: 13 - DNA, 21 bp, synthetic construct
  misc_feature 1..19: note = RNA
  misc_feature 20..21: note = DNA
  source 1..21: mol_type = other DNA
ttttgggactaccaactcatt

SEQ ID NO: 14 - DNA, 21 bp, synthetic construct
  misc_feature 1..19: note = RNA
  misc_feature 20..21: note = DNA
  source 1..21: mol_type = other DNA
tgagttggtagtcccaaaatt

SEQ ID NO: 15 - DNA, 21 bp, synthetic construct
  misc_feature 1..19: note = RNA
  misc_feature 20..21: note = DNA
  source 1..21: mol_type = other DNA
cctcaaagattttgggacttt

SEQ ID NO: 16 - DNA, 21 bp, synthetic construct
  misc_feature 1..19: note = RNA
  misc_feature 20..21: note = DNA
  source 1..21: mol_type = other DNA
agtcccaaaatctttgaggtt

SEQ ID NO: 17 - DNA, 21 bp, synthetic construct
  misc_feature 1..19: note = RNA
  misc_feature 20..21: note = DNA
  source 1..21: mol_type = other DNA
tgactatgtcatattcacttt

SEQ ID NO: 18 - DNA, 21 bp, synthetic construct
  misc_feature 1..19: note = RNA
  misc_feature 20..21: note = DNA
  source 1..21: mol_type = other DNA
agtgaatatgacatagtcatt

SEQ ID NO: 19 - DNA, 21 bp, synthetic construct
  misc_feature 1..19: note = RNA
  misc_feature 20..21: note = DNA
  source 1..21: mol_type = other DNA
accactgaaacagctcacttt

SEQ ID NO: 20 - DNA, 21 bp, synthetic construct
  misc_feature 1..19: note = RNA
  misc_feature 20..21: note = DNA
  source 1..21: mol_type = other DNA
agtgagctgtttcagtggttt

SEQ ID NO: 21 - DNA, 21 bp, synthetic construct
  misc_feature 1..19: note = RNA
  misc_feature 20..21: note = DNA
  source 1..21: mol_type = other DNA
ctgacctttggatggtgcttt

SEQ ID NO: 22 - DNA, 21 bp, synthetic construct
  misc_feature 1..19: note = RNA
  misc_feature 20..21: note = DNA
  source 1..21: mol_type = other DNA
agcaccatccaaaggtcagtt

SEQ ID NO: 23 - RNA, 19 nt, synthetic construct
  source 1..19: mol_type = other RNA
agatggagagccttgtccc

SEQ ID NO: 24 - RNA, 19 nt, synthetic construct
  source 1..19: mol_type = other RNA
gggacaaggctctccatct

SEQ ID NO: 25 - RNA, 19 nt, synthetic construct
  source 1..19: mol_type = other RNA
cgtggtaagagaattcctt

SEQ ID NO: 26 - RNA, 19 nt, synthetic construct
  source 1..19: mol_type = other RNA
aaggaattctcttaccacg

SEQ ID NO: 27 - RNA, 19 nt, synthetic construct
  source 1..19: mol_type = other RNA
gagaaaagctgtctttatt

SEQ ID NO: 28 - RNA, 19 nt, synthetic construct
  source 1..19: mol_type = other RNA
aataaagacagcttttctc

SEQ ID NO: 29 - RNA, 19 nt, synthetic construct
  source 1..19: mol_type = other RNA
cttactaaaggacctcatg

SEQ ID NO: 30 - RNA, 19 nt, synthetic construct
  source 1..19: mol_type = other RNA
catgaggtcctttagtaag

SEQ ID NO: 31 - RNA, 19 nt, synthetic construct
  source 1..19: mol_type = other RNA
attatcaaaacaatgtttt

SEQ ID NO: 32 - RNA, 19 nt, synthetic construct
  source 1..19: mol_type = other RNA
aaaacattgttttgataat

SEQ ID NO: 33 - RNA, 19 nt, synthetic construct
  source 1..19: mol_type = other RNA
atgtctgaagcaaaatgtt

SEQ ID NO: 34 - RNA, 19 nt, synthetic construct
  source 1..19: mol_type = other RNA
aacattttgcttcagacat

SEQ ID NO: 35 - RNA, 19 nt, synthetic construct
  source 1..19: mol_type = other RNA
ttttgggactaccaactca

SEQ ID NO: 36 - RNA, 19 nt, synthetic construct
  source 1..19: mol_type = other RNA
tgagttggtagtcccaaaa

SEQ ID NO: 37 - RNA, 19 nt, synthetic construct
  source 1..19: mol_type = other RNA
cctcaaagattttgggact

SEQ ID NO: 38 - RNA, 19 nt, synthetic construct
  source 1..19: mol_type = other RNA
agtcccaaaatctttgagg

SEQ ID NO: 39 - RNA, 19 nt, synthetic construct
  source 1..19: mol_type = other RNA
tgactatgtcatattcact

SEQ ID NO: 40 - RNA, 19 nt, synthetic construct
  source 1..19: mol_type = other RNA
agtgaatatgacatagtca

SEQ ID NO: 41 - RNA, 19 nt, synthetic construct
  source 1..19: mol_type = other RNA
accactgaaacagctcact

SEQ ID NO: 42 - RNA, 19 nt, synthetic construct
  source 1..19: mol_type = other RNA
agtgagctgtttcagtggt

SEQ ID NO: 43 - RNA, 19 nt, synthetic construct
  source 1..19: mol_type = other RNA
ctgacctttggatggtgct

SEQ ID NO: 44 - RNA, 19 nt, synthetic construct
  source 1..19: mol_type = other RNA
agcaccatccaaaggtcag

SEQ ID NO: 45 - DNA, 21 bp, synthetic construct
  misc_feature 1..19: note = RNA
  misc_feature 20..21: note = DNA
  source 1..21: mol_type = other DNA
gacataaaaacattgtttttt

SEQ ID NO: 46 - DNA, 21 bp, synthetic construct
  misc_feature 1..19: note = RNA
  misc_feature 20..21: note = DNA
  source 1..21: mol_type = other DNA
aaaacaatgtttttatgtctt

SEQ ID NO: 47 - DNA, 21 bp, synthetic construct
  misc_feature 1..19: note = RNA
  misc_feature 20..21: note = DNA
  source 1..21: mol_type = other DNA
aaacattgttttgataatatt

SEQ ID NO: 48 - DNA, 21 bp, synthetic construct
  misc_feature 1..19: note = RNA
  misc_feature 20..21: note = DNA
  source 1..21: mol_type = other DNA
tattatcaaaacaatgttttt

SEQ ID NO: 49 - DNA, 21 bp, synthetic construct
  misc_feature 1..19: note = RNA
  misc_feature 20..21: note = DNA
  source 1..21: mol_type = other DNA
gtatgttgagagcaaaatttt

SEQ ID NO: 50 - DNA, 21 bp, synthetic construct
  misc_feature 1..19: note = RNA
  misc_feature 20..21: note = DNA
  source 1..21: mol_type = other DNA
aattttgctctcaacatactt

SEQ ID NO: 51 - DNA, 21 bp, synthetic construct
  misc_feature 1..19: note = RNA
  misc_feature 20..21: note = DNA
  source 1..21: mol_type = other DNA
taatcatcaccctgtttaatt

SEQ ID NO: 52 - DNA, 21 bp, synthetic construct
  misc_feature 1..19: note = RNA
  misc_feature 20..21: note = DNA
  source 1..21: mol_type = other DNA
ttaaacagggtgatgattatt

SEQ ID NO: 53 - DNA, 21 bp, synthetic construct
  misc_feature 1..19: note = RNA
  misc_feature 20..21: note = DNA
  source 1..21: mol_type = other DNA
attttgcttcagacataaatt

SEQ ID NO: 54 - DNA, 21 bp, synthetic construct
  misc_feature 1..19: note = RNA
  misc_feature 20..21: note = DNA
  source 1..21: mol_type = other DNA
tttatgtctgaagcaaaattt

SEQ ID NO: 55 - DNA, 21 bp, synthetic construct
  misc_feature 1..19: note = RNA
  misc_feature 20..21: note = DNA
  source 1..21: mol_type = other DNA
ctacaagagatcgaaagtttt

SEQ ID NO: 56 - DNA, 21 bp, synthetic construct
  misc_feature 1..19: note = RNA
  misc_feature 20..21: note = DNA
  source 1..21: mol_type = other DNA
aactttcgatctcttgtagtt

SEQ ID NO: 57 - DNA, 21 bp, synthetic construct
  misc_feature 1..19: note = RNA
  misc_feature 20..21: note = DNA
  source 1..21: mol_type = other DNA
ctgtttcagtggtttgagttt

SEQ ID NO: 58 - DNA, 21 bp, synthetic construct
  misc_feature 1..19: note = RNA
  misc_feature 20..21: note = DNA
  source 1..21: mol_type = other DNA
actcaaaccactgaaacagtt

SEQ ID NO: 59 - DNA, 21 bp, synthetic construct
  misc_feature 1..19: note = RNA
  misc_feature 20..21: note = DNA
  source 1..21: mol_type = other DNA
ggtttgagtgaatatgacatt

SEQ ID NO: 60 - DNA, 21 bp, synthetic construct
  misc_feature 1..19: note = RNA
  misc_feature 20..21: note = DNA
  source 1..21: mol_type = other DNA
tgtcatattcactcaaacctt

SEQ ID NO: 61 - RNA, 19 nt, synthetic construct
  source 1..19: mol_type = other RNA
gacataaaaacattgtttt

SEQ ID NO: 62 - RNA, 19 nt, synthetic construct
  source 1..19: mol_type = other RNA
aaaacaatgtttttatgtc

SEQ ID NO: 63 - RNA, 19 nt, synthetic construct
  source 1..19: mol_type = other RNA
aaacattgttttgataata

SEQ ID NO: 64 - RNA, 19 nt, synthetic construct
  source 1..19: mol_type = other RNA
tattatcaaaacaatgttt

SEQ ID NO: 65 - RNA, 19 nt, synthetic construct
  source 1..19: mol_type = other RNA
gtatgttgagagcaaaatt

SEQ ID NO: 66 - RNA, 19 nt, synthetic construct
  source 1..19: mol_type = other RNA
aattttgctctcaacatac

SEQ ID NO: 67 - RNA, 19 nt, synthetic construct
  source 1..19: mol_type = other RNA
taatcatcaccctgtttaa

SEQ ID NO: 68 - RNA, 19 nt, synthetic construct
  source 1..19: mol_type = other RNA
ttaaacagggtgatgatta

SEQ ID NO: 69 - RNA, 19 nt, synthetic construct
  source 1..19: mol_type = other RNA
attttgcttcagacataaa

SEQ ID NO: 70 - RNA, 19 nt, synthetic construct
  source 1..19: mol_type = other RNA
tttatgtctgaagcaaaat

SEQ ID NO: 71 - RNA, 19 nt, synthetic construct
  source 1..19: mol_type = other RNA
ctacaagagatcgaaagtt

SEQ ID NO: 72 - RNA, 19 nt, synthetic construct
  source 1..19: mol_type = other RNA
aactttcgatctcttgtag

SEQ ID NO: 73 - RNA, 19 nt, synthetic construct
  source 1..19: mol_type = other RNA
ctgtttcagtggtttgagt

SEQ ID NO: 74 - RNA, 19 nt, synthetic construct
  source 1..19: mol_type = other RNA
actcaaaccactgaaacag

SEQ ID NO: 75 - RNA, 19 nt, synthetic construct
  source 1..19: mol_type = other RNA
ggtttgagtgaatatgaca

SEQ ID NO: 76 - RNA, 19 nt, synthetic construct
  source 1..19: mol_type = other RNA
tgtcatattcactcaaacc

SEQ ID NO: 77 - DNA, 584 bp, synthetic construct
  source 1..584: mol_type = other DNA
gaaaaaagagaaaagctgtctttattgcaaaataaagacagcttttctccggatcttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcactagtatcaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctagcgtggtaagagaattccttttgcaaggaattctcttaccacgtttttt

(19) United States
(12) Patent Application Publication
(10) Pub. No.: US 2026/0347852 A1
(43) Pub. Date: Nov. 5, 2026

(54) ANTIVIRAL NUCLEIC ACIDS AND COMPOSITIONS

(71) Applicant: GRIFFITH UNIVERSITY, Nathan, Queensland (AU)

(72) Inventor: Kevin MORRIS, Queensland (AU)

(21) Appl. No.: 19/122,162

(22) PCT Filed: Oct. 17, 2023
(86) PCT No.: PCT/AU2023/051021

§ 371 Date: Apr. 17, 2025

(30) Foreign Application Priority Data
Oct. 17, 2022 (AU) 2022903051

(51) Int. Cl. C12N 15/113; A61K 9/00; A61K 9/51; A61K 31/713; A61P 31/14

(52) U.S. Cl. CPC C12N 15/1131; A61K 9/0043; A61K 9/5123; A61K 31/713; A61P 31/14; C12N 2310/14

(57) ABSTRACT

The present invention relates generally to compositions and methods for inhibiting the replication of coronaviruses and treating diseases caused by coronavirus infection. More specifically, the invention provides nucleic acids capable of inhibiting coronavirus (e.g. SARS-CoV-2) replication and their use in treating patients infected by the virus

### CROSS-REFERENCE TO RELATED APPLICATIONS

This application is a national stage entry filed under 35 U.S.C. § 371 of International Application No. PCT/AU2023/051021, filed on Oct. 17, 2023, which claims priority to Australian Application No. 2022903051, filed on Oct. 17, 2022, the contents of each of which are hereby incorporated by reference in their entireties.

### TECHNICAL FIELD

The present invention relates generally to compositions and methods for inhibiting the replication of coronaviruses and treating diseases caused by coronavirus infection. More specifically, the invention provides nucleic acids capable of inhibiting coronavirus (e.g. SARS-CoV-2) replication and their use in treating patients infected by the virus.

### BACKGROUND

SARS-CoV-2 is a positive-sense single-stranded RNA virus that is contagious in humans and other animals. It is causative of the coronavirus disease 2019 (COVID-19) which has significantly affected people worldwide. As of September 2022, conservative estimates place the number of SARS-CoV-2 human infections since the commencement of the current pandemic at over 610 million, with over 6.5 million people dying from COVID-19 disease (see WHO Coronavirus (COVID-19) Dashboard).

In humans, SARS-CoV-2 infection results in a range of outcomes with many people experiencing very mild or almost unrecognizable symptoms, and others presenting with medium to severe illness, with an estimated fatality rate of approximately 1% worldwide. Significant resources and effort have been allocated to preventative measures through vaccine development, but it is well-recognised that therapeutic measures are critical to reducing morbidity and mortality. In particular, antivirals suitable for administration to patients during the early stages of the disease offer an opportunity to reduce viral replication and to thereby inhibit the onset of severe illness, particularly in people in high risk categories (e.g. the immunocompromised, elderly and so forth).

A need exists for effective therapeutic treatments to counter COVID-19 disease, including those treatments capable of inhibiting viral replication during the early stages of COVID-19 disease.

### SUMMARY OF THE INVENTION

The present invention addresses a need in the field of therapeutic treatments for COVID-19 disease by providing nucleic acids capable of inhibiting SARS-CoV-2 expression.

Various aspects of the present invention relate to nucleic acids capable of inhibiting the replication of coronaviruses, such as SARS-CoV-2. These nucleic acids, and compositions including them, can be used to treat diseases arising from coronavirus infection including COVID-19.

In some embodiments, there is provided a nucleic acid comprising 15 to 30 nucleotides and capable of specifically hybridising to a SARS-CoV-2 sequence as defined in SEQ ID NO: 23, SEQ ID NO: 25, or SEQ ID NO: 27, or capable of hybridising to a fragment of said SARS-CoV-2 sequence. The nucleic acid may comprise or consist of: 15 to 25 nucleotides; 15 to 23 nucleotides; 15 to 22 nucleotides; 15 to 21 nucleotides; 15 to 20 nucleotides; 15 to 19 nucleotides; 19 nucleotides; 20 nucleotides; 21 nucleotides, 22 nucleotides or 23 nucleotides.

In other embodiments, there is provided a nucleic acid having at least 80%, at least 85%, at least 90%, at least 95% or at least 97% sequence identity to:
a sequence as defined in SEQ ID NO: 2 or 24, wherein the nucleic acid is capable of specifically hybridising to a SARS-CoV-2 sequence as defined in SEQ ID NO: 23; or
a sequence as defined in SEQ ID NO: 4 or 26, wherein the nucleic acid is capable of specifically hybridising to a SARS-CoV-2 sequence as defined in SEQ ID NO: 25; or
a sequence as defined in SEQ ID NO: 6 or 28, wherein the nucleic acid is capable of specifically hybridising to a SARS-CoV-2 sequence as defined in SEQ ID NO: 27.

In further embodiments, fragments of the nucleic acids of the invention are provided, wherein the fragments are 10, 11, 12, 13, 14, 15, 16, 17, or 18 nucleotides in length.

In still other embodiments, a double stranded nucleic acid for inhibiting expression of SARS-CoV-2 is provided, comprising a sense strand and an antisense strand, wherein:
the sense strand comprises a sequence as defined in SEQ ID NO: 1 or 23 or a variant or fragment thereof, and the antisense strand comprises a sequence as defined in SEQ ID NO: 2 or 24 or a variant or fragment thereof, or
the sense strand comprises a sequence as defined in SEQ ID NO: 3 or 25 or a variant or fragment thereof, and the antisense strand comprises a sequence as defined in SEQ ID NO: 4 or 26 or a variant or fragment thereof, or
the sense strand comprises a sequence as defined in SEQ ID NO: 5 or 27 or a variant or fragment thereof, and the antisense strand comprises a sequence as defined in SEQ ID NO: 6 or 28 or a variant or fragment thereof.

In further embodiments, the nucleic acids of the invention may comprise: a 2′-deoxy-2′-fluoro modified nucleotide; a 2′-deoxy-modified nucleotide; a locked nucleotide; an abasic nucleotide; 2′-amino-modified nucleotide; 2′-alkyl-modified nucleotide; morpholino nucleotide; a non-natural base comprising nucleotide; a 2′-O-methyl modified nucleotide; a 2′-O-methoxyethoxy modified nucleotide; a 2′-fluoro modified nucleotide; a 5-methyl-modified cytidine; pseudouridine; a nucleotide comprising a 5′-phosphorothioate group and a terminal nucleotide linked to a cholesteryl derivative or dodecanoic acid bisdecylamide group; a nucleotide comprising phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite; or a nucleotide comprising deoxyribose.

In other embodiments, the nucleic acids of the invention may be conjugated to a ligand.

In other embodiments, the nucleic acids of the invention may be RNA, antisense RNA, or siRNA.

Other aspects of the present invention provide cells comprising the nucleic acids described herein, vectors comprising a nucleic acid sequence that encodes RNA, antisense RNA, or siRNA described herein, lipid nanoparticles comprising the nucleic acids and/or vectors described herein, and pharmaceutical compositions comprising the nucleic acids, vectors, and/or lipid nanoparticles described herein.

In some embodiments, the lipid nanoparticles of the invention may comprise any one or more of: a non-cationic lipid, a cationic lipid, a conjugated lipid for preventing aggregation of the nanoparticle.

In other embodiments, the pharmaceutical compositions may be a liquid for intravenous administration, or an aerosol for intranasal administration.

Further aspects of the present invention provide methods for inhibiting coronavirus replication in cells, methods for treating coronavirus infection in a subject, and methods for treating COVID-19 disease in a subject.

In some embodiments there is provided a method for inhibiting replication of a coronavirus in a cell, the method comprising administering a nucleic acid, vector, lipid nanoparticle or pharmaceutical composition described herein to the cell, to thereby cause degradation of a coronavirus mRNA molecule in the cell and inhibit said replication of the coronavirus.

In other embodiments there is provided a method for treating a coronavirus infection in a subject, the method comprising administering a therapeutically effective amount of a nucleic acid, vector, lipid nanoparticle or pharmaceutical composition described herein to the subject, to thereby inhibit replication of the coronavirus and treat said infection.

In further embodiments there is provided a method for treating COVID-19 disease in a subject, the method comprising administering a therapeutically effective amount of a nucleic acid, vector, lipid nanoparticle or pharmaceutical composition described herein to the subject, to thereby inhibit replication of the coronavirus and treat said COVID-19 disease.

In some embodiments, the coronavirus is SARS-CoV-2.

Still other aspects of the present invention provide methods for preparing medicaments and medical uses for the various nucleic acids, vectors, lipid nanoparticles and pharmaceutical compositions described herein.

In some embodiments there is provided use of the nucleic acid, vector, lipid nanoparticle or pharmaceutical composition described herein in the preparation of a medicament for inhibiting replication of a coronavirus in a cell, treating a coronavirus infection in a subject, or treating COVID-19 disease in a subject.

In other embodiments there is provided the nucleic acid, vector, lipid nanoparticle or pharmaceutical composition described herein for use in inhibiting replication of a coronavirus in a cell, treating a coronavirus infection in a subject, or treating COVID-19 disease in a subject.

In some embodiments, the coronavirus is SARS-CoV-2.

### DEFINITIONS

As used in this application, the singular form “a”, “an” and “the” include plural references unless the context clearly dictates otherwise. For example, the term “cell” also includes multiple cells unless otherwise stated.

As used herein, the term “comprising” means “including”, in a non-exhaustive sense. Variations of the word “comprising”, such as “comprise” and “comprises” have correspondingly varied meanings. Thus, for example, a composition “comprising” a given component A may consist exclusively of component A, or may include one or more additional components such as component B. Similarly, a pharmaceutical composition “comprising” a given nucleic acid may include one or more additional components, for example, a pharmaceutically acceptable excipient, diluent and/or carrier.

As used herein, the term “about”, when used in reference to a recited numerical value, includes the recited numerical value and numerical values within plus or minus ten percent of the recited value.

As used herein, the term “SARS-CoV-2” refers to severe acute respiratory syndrome-associated coronavirus 2.

As used herein, the term “nucleic acid” refers to nucleotides and polymers of nucleotides.

The nucleotides contemplated include ribonucleotides, deoxyribonucleotides, modified versions thereof, and combinations thereof. The polymers of nucleotides may be in single stranded form or double stranded form, including single and double stranded RNA, single and double stranded DNA, and double stranded RNA/DNA hybrids. Non-limiting examples of nucleic acids include all forms of RNA such as, for example, siRNA, mRNA, miRNA, shRNA, antisense RNA, guide RNA, and dicer substrate RNA and siRNAs. All of these forms of RNA can functionally target and repress viruses. Other non-limiting examples of “nucleic acids” include all forms of DNA such as, for example, genomic DNA, complementary DNA (cDNA), minicircle DNA, and plasmid DNA. Also included within the scope of the term “nucleic acid” are those comprising nucleotide analogues, modified backbone linkages or residues and the like, which have a physical structure that is related to a DNA or RNA molecule or residue, and which may be capable of forming a hydrogen bond with a DNA or RNA residue or an analogue thereof (i.e. it is able to hybridise with a DNA or RNA residue or an analogue thereof to form a base-pair), thus having similar binding properties to the ribonucleotide or deoxyribonucleotide residue to which they are structurally related (i.e. base nucleotide), and which may be metabolised in similar way to the base nucleotide. Non-limiting examples of nucleic acid analogues include methylated, iodinated, brominated or biotinylated residues, and those with an alteration of the sugar component or phosphodiester backbone. Contemplated nucleic acid analogues also include, without limitation, those with modifications to the nucleotide bases such as for example pseudouridine, 2′-fluoro, 2′-O-methyl, 5-methyl cytidine, 2′-O-methoxyethoxy, and those with peptide nucleic acid backbones and linkages. Other analogue nucleic acids include those with modified sugars (e.g. deoxyribose), non-ionic backbones, positive backbones, and non-ribose backbones (e.g. locked nucleic acids or phosphorodiamidate morpholino oligos).

As used herein, term “RNA interference” refers to a mechanism of action imbued in mammalian cells that can specifically turn off the production of proteins in cells in a sequence-specific and potent manner. In the process RNA molecules are involved in sequence-specific suppression of gene expression by double-stranded RNA, through translational or transcriptional repression. By way of example, RNA interference can be initiated via the introduction of small-interfering RNAs (siRNA, 15-30 bp of double stranded RNA) that specifically target mRNAs via sequence complementarity, causing their subsequent degradation. Additionally, small hairpin RNAs (shRNA) can also repress transcript and gene expression.

As used herein, an “antisense” nucleic acid or sequence is one that this complementary to at least a component of a specific target nucleic acid facilitating hybridisation with the target (for example under physiological conditions), thereby inhibiting biological activities involving the target, non-limiting examples of which include inhibiting translation of the target nucleic acid, altering transcript splicing, and the like. Antisense nucleic acids will hybridise specifically to their nucleic acid target meaning that they have a greater propensity to hybridise to their nucleic acid target, for example under physiological conditions, as compared to other non-target nucleic acids. Antisense nucleic acids can include those comprising nucleotide analogues, modified backbone linkages or residues and the like.

As used herein, the terms “siRNA” and “small interfering RNA” refers to a single stranded or double stranded RNA (ribonucleic acid) with the capacity to inhibit the expression of a given target nucleic acid when present in a cell comprising the target nucleic acid, by hybridising to the target nucleic acid and thereby inhibiting its standard biological activity (for example, its translation into a protein). The target nucleic acid may be a single stranded or double stranded RNA, a single stranded or double stranded DNA, and non-limiting examples include messenger RNA (mRNA) and promoter sequences. A single stranded or double stranded siRNA may typically be 15-50 nucleotides in length. siRNAs can functionally target and repress viruses.

As used herein, the terms “shRNA” and “short hairpin RNA” refers to an artificial RNA molecule with a tight hairpin turn that can be used to silence target gene expression via RNA interference (RNAi). shRNAs comprise a stem region of paired antisense and sense strands connected by unpaired nucleotides that make up a loop, similar to endogenous micro RNAs. The skilled person will appreciate that shRNA, once delivered to a cell, can load into the RNA-induced silencing complex (RISC), which degrades the passenger sense strand. The antisense (guide) strand directs RISC to mRNA that has a complementary sequence for degradation. Thus, shRNAs can functionally target and repress viruses and are functionally equivalent to siRNAs.

As used herein, the term “loop” or “loop region” is a sequence that joins two complementary strands of nucleic acid. In certain embodiments, the loop region is from 4-20 nucleotides in length, such as 15-19 nucleotides in length. From 0-50% of the loop region can be complementary to another portion of the loop region. The nucleotide sequence of the loop region may vary and could be, for example, (5′-GCAA-3′), (5′-GCGC-3′) or (5′-TTGC-3′) or other sequences as will be well understood by the skilled person. As used herein the terms “hybridise” and “hybridisation” refer to the binding of nucleic acid strands by complementary base pairing and include scenarios of binding based on partial complementarity or based on full complementarity. As known to those of ordinary skill in the art, the degree of hybridisation between two nucleic acid strands can be affected by parameters such as temperature, salt concentration and the like, and conditions under which two complementary or partially complementary nucleic acid strands will hybridise in a specific manner and avoid hybridising with other potential binding partners can be readily determined using standard optimisation.

As used herein, the term “variant” as refers to a substantially similar nucleic acid or polypeptide sequence. Generally, sequence variants possess qualitative biological activity in common. Further, such sequence variants may share at least 50%, 55%, 60%, 65%, 70%, 75%, 80%, 85%, 90%, 95%, 96%, 97%, 98% or 99% sequence identity over the full length of a reference sequence, or over a specified region of the reference sequence. Also included within the meaning of the term “variant” are homologues, which are typically a polypeptide or nucleic acid from a different species but sharing substantially the same biological function or activity as the corresponding polypeptide or nucleic acid disclosed herein.

As used herein, a percentage of “sequence identity” will be understood to arise from a comparison of two sequences in which they are aligned to give a maximum correlation between the sequences. This may include inserting “gaps” in either one or both sequences to enhance the degree of alignment. The percentage of sequence identity may then be determined over the length of each of the sequences or portions thereof being compared. For example, a nucleotide sequence (“subject sequence”) having at least 95% “sequence identity” with another nucleotide sequence (“query sequence”) is intended to mean that the subject sequence is identical to the query sequence except that the subject sequence may include up to five nucleotide alterations per 100 nucleotides of the query sequence. In other words, to obtain a nucleotide sequence of at least 95% sequence identity to a query sequence, up to 5% (i.e. 5 in 100) of the nucleotides in the subject sequence may be inserted or substituted with another nucleotide or deleted. The percentage of sequence identity between two sequences may be determined by comparing two optimally aligned sequences over a comparison window. A portion of a sequence in the comparison window may, for example, comprise deletions or additions (i.e. gaps) in comparison to a reference sequence (e.g. one derived from another species) which does not comprise deletions or additions, in order to align the two sequences optimally, or vice versa. A percentage of sequence identity may then be calculated by determining the number of positions at which identical nucleotides occur in both sequences to yield the number of matched positions, dividing the number of matched positions by the total number of positions in the window of comparison and multiplying the result by 100 to yield the percentage of sequence identity. In the context of two or more nucleic acid sequences, the percentage of sequence identity refers to the specified percentage of nucleotides that are the same over a specified region (or, when not specified, over the entire sequence) when compared and aligned for maximum correspondence over a comparison window, or designated region as measured using one of the following sequence comparison algorithms or by manual alignment and visual inspection. For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated.

The sequence comparison algorithm then calculates the percentage of sequence identity for the test sequence(s) relative to the reference sequence, based on the program parameters. Methods of alignment of sequences for comparison are known in the art. Optimal alignment of sequences for determination of sequence identity can be achieved conventionally using known computer programs, including, but not limited to: CLUSTAL in the PC/Gene program (available from Intelligenetics, Mountain View, California); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, California, USA).

Alignments using these programs can be performed using the default parameters. Another method for determining the best overall match between a query sequence and a subject sequence, also referred to as a global sequence alignment, can be determined using the FASTDB computer program based on the algorithm of Brutlag and colleagues (Comp. App. Biosci. 6:237-245 (1990)).

As used herein, the term “fragment” in relation to a nucleic acid refers to a constituent of that nucleic acid. Typically, the fragment possesses qualitative biological activity in common with the nucleic acid, which may comprise, for example, hybridisation to another target nucleic acid causing expression of the target nucleic acid to be reduced, inhibited and the like. Fragments may be derived from a nucleic acid of the invention or alternatively may be synthesized by some other means, for example chemical synthesis.

As used herein, the term “isolated” in the context of a nucleic acid or other biological entity will be understood to mean that the isolated nucleic acid or other biological entity is at least partially free of and in some cases free or substantially free of nucleic acids, proteins, lipids, carbohydrates or other materials which normally accompany it as found in its natural/native state. An “isolated” nucleic acid or other biological entity may be purified so as to facilitate a partial or full separation from other biological entities which normally accompany it as found in its natural/native state.

As used herein, the terms “treat”, “treating”, “treatment”, and the like refer to reducing or ameliorating a disorder/disease and/or symptoms associated therewith. It will be appreciated, although not precluded, that treating a disorder or condition does not necessarily require that the disorder, condition, or symptoms associated therewith are completely eliminated. However, it is contemplated that the disorder, condition, or symptoms associated therewith are improved as compared to prior to the treatment commencing.

As used herein, the term “subject” includes any animal of economic, social or research importance including bovine, equine, ovine, primate, avian and rodent species. Hence, a “subject” may be a mammal such as, for example, a human or a non-human mammal.

As used herein, a “therapeutically effective amount” of a given composition or similar under administration to a subject will be understood to be an amount of the therapeutic agent that is sufficient to partially or completely ameliorate the condition or disease under treatment, including for example, a reduction of a symptom(s) of the condition or disease, such as by decreasing the severity or frequency of the symptom(s), or eliminating the symptom(s). For example, in relation to a given symptom, a therapeutically effective amount may result in a reduction of least 5%, 10%, 15%, 20%, 25%, 40%, 50%, 60%, 75%, 80%, 90%, or 100% of the symptom. The actual “therapeutically effective amount” will depend on the specific composition being administered, the condition or disease under treatment, the age and health of the subject under treatment, and so forth. Such parameters are routine to assess and can readily be determined by those of ordinary skill in the art.

### BRIEF DESCRIPTION OF THE FIGURES

Preferred embodiments of the present invention will now be described by way of example only, with reference to the accompanying figures wherein:

FIG. 1 shows the results of an interferon screen of SARS-CoV-2 targeted new candidate siRNAs. Error bars denote SEM of triplicate treatments.

FIG. 2 provides the results of a plaque assay screen and assessment of new candidate siRNAs for repression of SARS-CoV-2 in vitro. Data are shown with the standard error of the mean of triplicate treatments and *p<0.05, ***p<0.01, and ****p<0.001 were considered statistically significant as determined by one-way ANOVA analysis (Dunnett's post-test) when compared against N367S (control).

FIG. 3 shows the results of a qRT-PCR screen of new candidate siRNAs.

FIG. 4 provides the results of a plaque assay screen and assessment of new candidate siRNAs for repression of SARS-CoV-2 in vitro. Data shown is % plaque inhibition of average plaque counts for each treatment relative to virus alone.

FIG. 5 provides the results of another plaque assay screen and assessment of selected candidate siRNAs for repression of Alpha, Beta, Kappa, Delta or Omicron variant SARS-CoV-2 VOCs in vitro. Data was collected from triplicate treatments and are shown with the standard error of the mean of triplicate treatments and *p<0.05, ***p<0.01, and ****p<0.001 were considered statistically significant as determined by one-way ANOVA analysis (Dunnett's post-test) when compared against N367S (control).

FIG. 6 shows the results of a further plaque assay screen and an assessment of new candidate negative strand targeting siRNAs for repression of SARS-CoV-2 Delta variant in vitro. Data was collected from triplicate treatments and are shown with the standard error of the mean of triplicate treatments and *p<0.05, ***p<0.01, and ****p<0.001 were considered statistically significant as determined by one-way ANOVA analysis (Dunnett's post-test) when compared against N367S (control).

FIG. 7 provides the results of an additional plaque assay screen and assessment of combination of new candidate negative strand and positive strand targeting siRNAs for repression of SARS-CoV-2 Wuhan and Delta variant in vitro. Data was collected from triplicate treatments and are shown with the standard error of the mean of triplicate treatments and *p<0.05, ***p<0.01, and ****p<0.001 were considered statistically significant as determined by one-way ANOVA analysis (Dunnett's post-test) when compared against N367S (control). % viral plaque inhibition by the siRNAs is also shown.

FIG. 8 provides the results of a dose response evaluation of top three candidate siRNAs for repression of Delta variant in vitro. Data was collected from triplicate treatments and are shown with the standard error of the mean of triplicate treatments reflected on each bar graph.

FIG. 9 provides A) a timeline for viral infection then treatment from day −1 to day 3 with a daily dose of 1 mg/kg siRNA being administered on each day from day 0 and all mice being culled on day 3 and B) an in vivo evaluation of HelUP2 siRNAs delivered by stealth LNPs. Lung viral tissue counts/g at 3 dpi are shown. Each dot represents data from one mouse. ** p<0.005 one-way ANOVA.

FIG. 10 shows the results of an experiment in which Vero E6 cells were transfected with siControl and siCoV_1 (30 nM) complexed to Lipofectamine 2000 for 24 h before infecting with delta SARS-CoV-2 VOC at 250 plaque forming unit (PFU). Infectious viral plaques were counted 4 days post infection (dpi). Data is expressed as mean percentage plaque inhibition relative to virus alone (control) and is representative of the standard error of the mean (SEM) of triplicate treatments. One-way ANOVA (Dunnett's post-test) was done and compared against siControl.

FIG. 11 shows the results of an experiment in which Vero E6 cells were transfected with increasing concentrations (0.1-30 nM) of siControl and siCoV_1 complexed to Lipofectamine 2000 for 24 h before infecting with delta SARS-CoV-2 VOC at 250 PFU. Infectious viral plaques were counted 4 dpi. Data is expressed as mean plaque counts and standard error of the mean (SEM) of triplicate treatments. One-way ANOVA (Dunnett's post-test) was done and compared against siControl.

FIG. 12 provides A) a schematic summary of in vivo testing approach and siRNA treatment regimens and B) K18 h-ACE2 mice infected with 1×10⁵ live delta SARS-CoV-2 VOC received either daily intravenous (IV) (retro-orbital, sLNP) or intranasal (IN, dmLNP) treatment of siRNA-LNP (1 mg/kg) daily (−1 to 2 dpi). The control siRNA used here is siN367 (siControl). Tissue viral tissue counts/g at 3 dpi are shown. Each dot represents data from one mouse and bars represent the mean. One-way ANOVA (Dunnett's post-test) was performed against siControl. Mice were weighted daily, and data points denote mean percentage weight gain and error bars represent SEM. Two-way ANOVA was performed against siControl at each time point.

FIG. 13 is a schematic diagram depicting a dual-shRNA expressing cassette to be used for expressing SARS-CoV-2 shRNAs to be packaged into EVs. Both H1 and U6 promoters are shown here, but either U6 or H1 promoters and shRNAs can be expressed alone, or as in the case here, in combination.

### DETAILED DESCRIPTION

The following detailed description conveys exemplary embodiments of the present invention in sufficient detail to enable those of ordinary skill in the art to practice the present invention. Features or limitations of the various embodiments described do not necessarily limit other embodiments of the present invention, or the present invention as a whole. Hence, the following detailed description does not limit the scope of the present invention, which is defined only by the claims.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

### Inhibitory Nucleic Acids

The present invention provides nucleic acids designed to inhibit the expression of target genes and/or inhibit the biological activity of non-translated target sequences (e.g. regulatory sequences).

Without limitation, genes and regulatory sequences targeted by the inhibitory nucleic acids described herein may derive from a pathogen causative of a disease or condition upon infecting a host organism. For example, the pathogen may be a virus capable of infecting mammalian subjects, including a human.

The virus may be an RNA virus such as, for example, a coronavirus. Non limiting examples include alpha coronaviruses including the human coronaviruses HCoV-229E and HCoV-NL63, and beta coronaviruses including the human coronaviruses SARS-CoV-1 (causative of severe acute respiratory syndrome), SARS-CoV-2 (causative of coronavirus disease 2019/COVID-19), MERS-CoV (causative of Middle East respiratory syndrome), HCoV-HKU1 and HCoV-OC43.

The inhibitory nucleic acids described herein may inhibit the expression of viral protein(s), including coronavirus proteins. By way of non-limiting example, the inhibitory nucleic acids may inhibit the expression of a structural coronavirus protein (e.g. a spike, membrane, envelope or nucleocapsid protein). The protein may be essential for viral replication such as, for example, a helicase protein. Additionally or alternatively, the inhibitory nucleic acids may inhibit the expression of a non-structural coronavirus protein. Additionally or alternatively, the inhibitory nucleic acids may inhibit the expression of the ORF1ab and/or the polymerase gene.

The inhibitory nucleic acids described herein may inhibit the biological activity of untranslated segments of viral genomes. For example, they may inhibit the biological activity of stem loop structures in the 5′ or 3′ untranslated region (i.e. 5′ UTR or 3′ UTR) of a coronavirus (e.g. stem-loop 5 (SL5) of a coronavirus 5′ UTR).

The inhibitory nucleic acids described herein may be single stranded or double stranded (including but not limited to hairpin structures).

The inhibitory nucleic acids described herein may have a length of between: 15 and 30 nucleotides, 15 and 25 nucleotides, 15 and 20 nucleotides, 18 and 25 nucleotides, or 25 and 30 nucleotides, or 18, 19, 20, 21, 22, 23, 24, or 25 nucleotides.

The double stranded inhibitory nucleic acids may be provided in a form where each strand of the duplex is the same length or a different length. The double stranded inhibitory nucleic acids may thus comprise one or more single strand overhangs of at least 1, 2, 3 or 4 nucleotides, 1-2 nucleotides, 1-4 nucleotides, 2-4 nucleotides, 2-5 nucleotides, 1-10 nucleotides, 2-10 nucleotides, or 5-10 nucleotides. The overhang(s) may be provided at the 5′ and/or 3′ end of the sense strain, the 5′ and/or 3′ end of the antisense strain, the 5′ end of the sense strain and the 5′ end of the antisense strain, or the 3′ end of the sense strain and the 3′ end of the antisense strain.

The inhibitory nucleic acids described herein (e.g. variants of the inhibitory nucleic acids) may contain one or more mismatches to a target sequence (e.g. 1, 2, 3, 4 or 5 mismatched nucleotides, or less than 5, less than 4, or less than 3 mismatched nucleotides). The mismatches may be located 5′ and/or 3′ to a central portion of the nucleic acid, including for example, within 6, 5, 4, 3 or 2 nucleotides of its 3′ and/or 5′ terminus. For example, in the case of the nucleic acid sequences defined by any of SEQ ID NOs: 1-20 or 23-42, a mismatch with their respective target sequences may not be present in the central 9, 10, 11, 14, 14, 15, 16, 17 or 18 nucleotides of SEQ ID NOs: 1-20 or 23-42. Based on the methods described herein and common general knowledge in the art, persons of ordinary skill can readily determine whether a given variant of an inhibitory nucleic acid described herein containing mismatched nucleotide(s) to its target sequence will still be effective in inhibiting the expression of a target gene and/or the biological activity of non-translated target sequences (e.g. regulatory sequences). Additionally, persons of ordinary skill will appreciate that that there is no substantial difference in the potency of siRNA made with different overhangs and that the composition of the overhang does not appear to play a critical role in target mRNA recognition and cleavage. The siRNAs may have dTdT overhangs, but could equally have UU or AA overhangs or another overhang such as an overhang complementary to the mRNA sequence, or the overhang could be absent.

The inhibitory nucleic acids described herein may comprise one or more modifications such as, for example, modified backbones, substitution of internucleoside linkages, substitution of sugar components, nucleobases and the like. Typically, and although not a requirement, the modifications may improve one or more performance parameters of the nucleic acid (e.g. stability, strength of hybridisation to a target sequence, provide simpler/more cost-effective manufacture). Persons of ordinary skill in the field are well aware of the various nucleic acid modifications available, their benefits, and how to introduce them into a reference sequence.

The inhibitory nucleic acids described herein can be manufactured using standard methods known in the art, including for example, those described in “Current protocols in nucleic acid chemistry,” Beaucage, S. L. et al. (Eds.), John Wiley & Sons, Inc., New York, N.Y., USA, the entire content of which is incorporated herein by cross-reference. Suitable and non-limiting chemical synthesis methods for generating nucleic acids without modifications include procedures utilising common nucleic acid such as phosphoramidites at the 3′ terminus and dimethoxytrityl at the 5′ terminus (as described, for example, in Usman et al., 1987, J. Am. Chem. Soc., 109, 7845; Scaringe et al., 1990, Nucleic Acids Res., 18, 5433). Taking the specific example of inhibitory nucleic acids (e.g. siRNA), non-limiting methods include those involving synthesis, deprotection and analysing as set out, for example, in U.S. Pat. Nos. 6,649,751, 6,673,918, 6,686,463, 6,989,442 and 6,995,259. Alternatively, they may be synthesised separately and combined together post synthetically, for example, by ligation (see Bellon et al., 1997, Nucleosides & Nucleotides, 16, 951; Bellon et al., 1997, Bioconjugate Chem. 8, 204; International PCT Publication No. WO 93/23569; Shabarova et al., 1991, Nucleic Acids Research 19, 4247; Moore et al., 1992, Science 256, 9923), or by hybridisation following synthesis and/or deprotection. They may also be synthesised as described in U.S. Pat. Nos. 5,889,136; 6,008,400 and 6,111,086.

In certain aspects of the present invention, inhibitory nucleic acids are provided comprising or consisting of the sequence defined in SEQ ID NO: 1 or 23, or variants or fragments of the sequence defined in SEQ ID NO: 1 or 23. The variants may comprise or consist of a sequence having at least 80%, at least 85%, at least 90%, at least 95% or at least 97% sequence identity to the sequence defined in SEQ ID NO: 1 or 23. Where an overhang is specified, the variants may include different overhangs. The fragments may comprise or consist of a 10, 11, 12, 13, 14, 15, 16, 17, or 18 nucleotide fragment of the sequence defined in SEQ ID NO: 1 or 23.

In other aspects of the present invention, inhibitory nucleic acids are provided comprising or consisting of the sequence defined in SEQ ID NO: 2 or 24, or variants or fragments of the sequence defined in SEQ ID NO: 2 or 24. The variants may comprise or consist of a sequence having at least 80%, at least 85%, at least 90%, at least 95% or at least 97% sequence identity to the sequence defined in SEQ ID NO: 2 or 24. Where an overhang is specified, the variants may include different overhangs. The fragments may comprise or consist of a 10, 11, 12, 13, 14, 15, 16, 17, or 18 nucleotide fragment of the sequence defined in SEQ ID NO: 2 or 24.

In certain aspects of the present invention, inhibitory nucleic acids are provided comprising or consisting of the sequence defined in SEQ ID NO: 3 or 25, or variants or fragments of the sequence defined in SEQ ID NO: 3 or 25. The variants may comprise or consist of a sequence having at least 80%, at least 85%, at least 90%, at least 95% or at least 97% sequence identity to the sequence defined in SEQ ID NO: 3 or 25. Where an overhang is specified, the variants may include different overhangs. The fragments may comprise or consist of a 10, 11, 12, 13, 14, 15, 16, 17, or 18 nucleotide fragment of the sequence defined in SEQ ID NO: 3 or 25.

In other aspects of the present invention, inhibitory nucleic acids are provided comprising or consisting of the sequence defined in SEQ ID NO: 4 or 26, or variants or fragments of the sequence defined in SEQ ID NO: 4 or 26. The variants may comprise or consist of a sequence having at least 80%, at least 85%, at least 90%, at least 95% or at least 97% sequence identity to the sequence defined in SEQ ID NO: 4 or 26. Where an overhang is specified, the variants may include different overhangs. The fragments may comprise or consist of a 10, 11, 12, 13, 14, 15, 16, 17, or 18 nucleotide fragment of the sequence defined in SEQ ID NO: 4 or 26.

In certain aspects of the present invention, inhibitory nucleic acids are provided comprising or consisting of the sequence defined in SEQ ID NO: 5 or 27, or variants or fragments of the sequence defined in SEQ ID NO: 5 or 27. The variants may comprise or consist of a sequence having at least 80%, at least 85%, at least 90%, at least 95% or at least 97% sequence identity to the sequence defined in SEQ ID NO: 5 or 27. Where an overhang is specified, the variants may include different overhangs. The fragments may comprise or consist of a 10, 11, 12, 13, 14, 15, 16, 17, or 18 nucleotide fragment of the sequence defined in SEQ ID NO: 5 or 27.

In other aspects of the present invention, inhibitory nucleic acids are provided comprising or consisting of the sequence defined in SEQ ID NO: 6 or 28, or variants or fragments of the sequence defined in SEQ ID NO: 6 or 28. The variants may comprise or consist of a sequence having at least 80%, at least 85%, at least 90%, at least 95% or at least 97% sequence identity to the sequence defined in SEQ ID NO: 6 or 28. Where an overhang is specified, the variants may include different overhangs. The fragments may comprise or consist of a 10, 11, 12, 13, 14, 15, 16, 17, or 18 nucleotide fragment of the sequence defined in SEQ ID NO: 6 or 28.

In certain aspects of the present invention, inhibitory nucleic acids are provided comprising or consisting of the sequence defined in SEQ ID NO: 7 or 29, or variants or fragments of the sequence defined in SEQ ID NO: 7 or 29. The variants may comprise or consist of a sequence having at least 80%, at least 85%, at least 90%, at least 95% or at least 97% sequence identity to the sequence defined in SEQ ID NO: 7 or 29. Where an overhang is specified, the variants may include different overhangs. The fragments may comprise or consist of a 10, 11, 12, 13, 14, 15, 16, 17, or 18 nucleotide fragment of the sequence defined in SEQ ID NO: 7 or 29.

In other aspects of the present invention, inhibitory nucleic acids are provided comprising or consisting of the sequence defined in SEQ ID NO: 8 or 30, or variants or fragments of the sequence defined in SEQ ID NO: 8 or 30. The variants may comprise or consist of a sequence having at least 80%, at least 85%, at least 90%, at least 95% or at least 97% sequence identity to the sequence defined in SEQ ID NO: 8 or 30. Where an overhang is specified, the variants may include different overhangs. The fragments may comprise or consist of a 10, 11, 12, 13, 14, 15, 16, 17, or 18 nucleotide fragment of the sequence defined in SEQ ID NO: 8 or 30.

In certain aspects of the present invention, inhibitory nucleic acids are provided comprising or consisting of the sequence defined in SEQ ID NO: 9 or 31, or variants or fragments of the sequence defined in SEQ ID NO: 9 or 31. The variants may comprise or consist of a sequence having at least 80%, at least 85%, at least 90%, at least 95% or at least 97% sequence identity to the sequence defined in SEQ ID NO: 9 or 31. Where an overhang is specified, the variants may include different overhangs. The fragments may comprise or consist of a 10, 11, 12, 13, 14, 15, 16, 17, or 18 nucleotide fragment of the sequence defined in SEQ ID NO: 9 or 31.

In other aspects of the present invention, inhibitory nucleic acids are provided comprising or consisting of the sequence defined in SEQ ID NO: 10 or 32, or variants or fragments of the sequence defined in SEQ ID NO: 10 or 32. The variants may comprise or consist of a sequence having at least 80%, at least 85%, at least 90%, at least 95% or at least 97% sequence identity to the sequence defined in SEQ ID NO: 10 or 32. Where an overhang is specified, the variants may include different overhangs. The fragments may comprise or consist of a 10, 11, 12, 13, 14, 15, 16, 17, or 18 nucleotide fragment of the sequence defined in SEQ ID NO: 10 or 32.

In certain aspects of the present invention, inhibitory nucleic acids are provided comprising or consisting of the sequence defined in SEQ ID NO: 11 or 33, or variants or fragments of the sequence defined in SEQ ID NO: 11 or 33. The variants may comprise or consist of a sequence having at least 80%, at least 85%, at least 90%, at least 95% or at least 97% sequence identity to the sequence defined in SEQ ID NO: 11 or 33. Where an overhang is specified, the variants may include different overhangs. The fragments may comprise or consist of a 10, 11, 12, 13, 14, 15, 16, 17, or 18 nucleotide fragment of the sequence defined in SEQ ID NO: 11 or 33. In other aspects of the present invention, inhibitory nucleic acids are provided comprising or consisting of the sequence defined in SEQ ID NO: 12 or 34, or variants or fragments of the sequence defined in SEQ ID NO: 12 or 34. The variants may comprise or consist of a sequence having at least 80%, at least 85%, at least 90%, at least 95% or at least 97% sequence identity to the sequence defined in SEQ ID NO: 12 or 34. Where an overhang is specified, the variants may include different overhangs. The fragments may comprise or consist of a 10, 11, 12, 13, 14, 15, 16, 17, or 18 nucleotide fragment of the sequence defined in SEQ ID NO: 12 or 34.

In certain aspects of the present invention, inhibitory nucleic acids are provided comprising or consisting of the sequence defined in SEQ ID NO: 13 or 35, or variants or fragments of the sequence defined in SEQ ID NO: 13 or 35. The variants may comprise or consist of a sequence having at least 80%, at least 85%, at least 90%, at least 95% or at least 97% sequence identity to the sequence defined in SEQ ID NO: 13 or 35. Where an overhang is specified, the variants may include different overhangs. The fragments may comprise or consist of a 10, 11, 12, 13, 14, 15, 16, 17, or 18 nucleotide fragment of the sequence defined in SEQ ID NO: 13 or 35.

In other aspects of the present invention, inhibitory nucleic acids are provided comprising or consisting of the sequence defined in SEQ ID NO: 14 or 36, or variants or fragments of the sequence defined in SEQ ID NO: 14 or 36. The variants may comprise or consist of a sequence having at least 80%, at least 85%, at least 90%, at least 95% or at least 97% sequence identity to the sequence defined in SEQ ID NO: 14 or 36. Where an overhang is specified, the variants may include different overhangs. The fragments may comprise or consist of a 10, 11, 12, 13, 14, 15, 16, 17, or 18 nucleotide fragment of the sequence defined in SEQ ID NO: 14 or 36.

In certain aspects of the present invention, inhibitory nucleic acids are provided comprising or consisting of the sequence defined in SEQ ID NO: 15 or 37, or variants or fragments of the sequence defined in SEQ ID NO: 15 or 37. The variants may comprise or consist of a sequence having at least 80%, at least 85%, at least 90%, at least 95% or at least 97% sequence identity to the sequence defined in SEQ ID NO: 15 or 37. Where an overhang is specified, the variants may include different overhangs. The fragments may comprise or consist of a 10, 11, 12, 13, 14, 15, 16, 17, or 18 nucleotide fragment of the sequence defined in SEQ ID NO: 15 or 37.

In other aspects of the present invention, inhibitory nucleic acids are provided comprising or consisting of the sequence defined in SEQ ID NO: 16 or 38, or variants or fragments of the sequence defined in SEQ ID NO: 16 or 38. The variants may comprise or consist of a sequence having at least 80%, at least 85%, at least 90%, at least 95% or at least 97% sequence identity to the sequence defined in SEQ ID NO: 16 or 38. Where an overhang is specified, the variants may include different overhangs. The fragments may comprise or consist of a 10, 11, 12, 13, 14, 15, 16, 17, or 18 nucleotide fragment of the sequence defined in SEQ ID NO: 16 or 38.

In certain aspects of the present invention, inhibitory nucleic acids are provided comprising or consisting of the sequence defined in SEQ ID NO: 17 or 39, or variants or fragments of the sequence defined in SEQ ID NO: 17 or 39. The variants may comprise or consist of a sequence having at least 80%, at least 85%, at least 90%, at least 95% or at least 97% sequence identity to the sequence defined in SEQ ID NO: 17 or 39. Where an overhang is specified, the variants may include different overhangs. The fragments may comprise or consist of a 10, 11, 12, 13, 14, 15, 16, 17, or 18 nucleotide fragment of the sequence defined in SEQ ID NO: 17 or 39.

In other aspects of the present invention, inhibitory nucleic acids are provided comprising or consisting of the sequence defined in SEQ ID NO: 18 or 40, or variants or fragments of the sequence defined in SEQ ID NO: 18 or 40. The variants may comprise or consist of a sequence having at least 80%, at least 85%, at least 90%, at least 95% or at least 97% sequence identity to the sequence defined in SEQ ID NO: 18 or 40. Where an overhang is specified, the variants may include different overhangs. The fragments may comprise or consist of a 10, 11, 12, 13, 14, 15, 16, 17, or 18 nucleotide fragment of the sequence defined in SEQ ID NO: 18 or 40.

In certain aspects of the present invention, inhibitory nucleic acids are provided comprising or consisting of the sequence defined in SEQ ID NO: 19 or 41, or variants or fragments of the sequence defined in SEQ ID NO: 19 or 41. The variants may comprise or consist of a sequence having at least 80%, at least 85%, at least 90%, at least 95% or at least 97% sequence identity to the sequence defined in SEQ ID NO: 19 or 41. Where an overhang is specified, the variants may include different overhangs. The fragments may comprise or consist of a 10, 11, 12, 13, 14, 15, 16, 17, or 18 nucleotide fragment of the sequence defined in SEQ ID NO: 19 or 41.

In other aspects of the present invention, inhibitory nucleic acids are provided comprising or consisting of the sequence defined in SEQ ID NO: 20 or 42, or variants or fragments of the sequence defined in SEQ ID NO: 20 or 42. The variants may comprise or consist of a sequence having at least 80%, at least 85%, at least 90%, at least 95% or at least 97% sequence identity to the sequence defined in SEQ ID NO: 20 or 42. Where an overhang is specified, the variants may include different overhangs. The fragments may comprise or consist of a 10, 11, 12, 13, 14, 15, 16, 17, or 18 nucleotide fragment of the sequence defined in SEQ ID NO: 20 or 42. In further aspects of the present invention, inhibitory nucleic acids are provided comprising or consisting of a first nucleic acid strand hybridised by complementary base pairing to a second nucleic acid strand. In some embodiments, the first and second strands may be provided in a hairpin structure. In other embodiments, the first and second strands are not provided in a hairpin structure (i.e. they each terminate discretely at their 5′ and 3′ termini). Either or both strands may have an overhang of unhybridized nucleotides at their 3′ end (e.g. 1, 2, 3, 4, or 5 unhybridized nucleotides). Alternatively, there may be no overhang sequence on either strand. The 5′ end of either or both strands may be phosphorylated and/or the 3′ end of either or both strands may be hydroxylated.

The first strand may comprise or consist of the sequence defined in SEQ ID NO: 1 or 23, or variants or fragments of the sequence defined in SEQ ID NO: 1 or 23. The variants may comprise or consist of a sequence having at least 80%, at least 85%, at least 90%, at least 95% or at least 97% sequence identity to the sequence defined in SEQ ID NO: 1 or 23. Where an overhang is specified, the variants may include different overhangs. The fragments may comprise or consist of a 10, 11, 12, 13, 14, 15, 16, 17, or 18 nucleotide fragment of the sequence defined in SEQ ID NO: 1 or 23. The second strand may comprise or consist of the sequence defined in SEQ ID NO: 2 or 24, or variants or fragments of the sequence defined in SEQ ID NO: 2 or 24. The variants may comprise or consist of a sequence having at least 80%, at least 85%, at least 90%, at least 95% or at least 97% sequence identity to the sequence defined in SEQ ID NO: 2 or 24. Where an overhang is specified, the variants may include different overhangs. The fragments may comprise or consist of a 10, 11, 12, 13, 14, 15, 16, 17, or 18 nucleotide fragment of the sequence defined in SEQ ID NO: 2 or 24.

Alternatively, the first strand may comprise or consist of the sequence defined in SEQ ID NO: 3 or 25, or variants or fragments of the sequence defined in SEQ ID NO: 3 or 25. The variants may comprise or consist of a sequence having at least 80%, at least 85%, at least 90%, at least 95% or at least 97% sequence identity to the sequence defined in SEQ ID NO: 3 or 25. Where an overhang is specified, the variants may include different overhangs. The fragments may comprise or consist of a 10, 11, 12, 13, 14, 15, 16, 17, or 18 nucleotide fragment of the sequence defined in SEQ ID NO: 3 or 25. The second strand may comprise or consist of the sequence defined in SEQ ID NO: 4 or 26, or variants or fragments of the sequence defined in SEQ ID NO: 4 or 26. The variants may comprise or consist of a sequence having at least 80%, at least 85%, at least 90%, at least 95% or at least 97% sequence identity to the sequence defined in SEQ ID NO: 4 or 26. Where an overhang is specified, the variants may include different overhangs. The fragments may comprise or consist of a 10, 11, 12, 13, 14, 15, 16, 17, or 18 nucleotide fragment of the sequence defined in SEQ ID NO: 4 or 26.

Alternatively, the first strand may comprise or consist of the sequence defined in SEQ ID NO: 5 or 27, or variants or fragments of the sequence defined in SEQ ID NO: 5 or 27. The variants may comprise or consist of a sequence having at least 80%, at least 85%, at least 90%, at least 95% or at least 97% sequence identity to the sequence defined in SEQ ID NO: 5 or 27. Where an overhang is specified, the variants may include different overhangs. The fragments may comprise or consist of a 10, 11, 12, 13, 14, 15, 16, 17, or 18 nucleotide fragment of the sequence defined in SEQ ID NO: 5 or 27. The second strand may comprise or consist of the sequence defined in SEQ ID NO: 6 or 28, or variants or fragments of the sequence defined in SEQ ID NO: 6 or 28. The variants may comprise or consist of a sequence having at least 80%, at least 85%, at least 90%, at least 95% or at least 97% sequence identity to the sequence defined in SEQ ID NO: 6 or 28. Where an overhang is specified, the variants may include different overhangs. The fragments may comprise or consist of a 10, 11, 12, 13, 14, 15, 16, 17, or 18 nucleotide fragment of the sequence defined in SEQ ID NO: 6 or 28.

Alternatively, the first strand may comprise or consist of the sequence defined in SEQ ID NO: 7 or 29, or variants or fragments of the sequence defined in SEQ ID NO: 7 or 29. The variants may comprise or consist of a sequence having at least 80%, at least 85%, at least 90%, at least 95% or at least 97% sequence identity to the sequence defined in SEQ ID NO: 7 or 29. Where an overhang is specified, the variants may include different overhangs. The fragments may comprise or consist of a 10, 11, 12, 13, 14, 15, 16, 17, or 18 nucleotide fragment of the sequence defined in SEQ ID NO: 7 or 29. The second strand may comprise or consist of the sequence defined in SEQ ID NO: 8 or 30, or variants or fragments of the sequence defined in SEQ ID NO: 8 or 30. The variants may comprise or consist of a sequence having at least 80%, at least 85%, at least 90%, at least 95% or at least 97% sequence identity to the sequence defined in SEQ ID NO: 8 or 30. Where an overhang is specified, the variants may include different overhangs. The fragments may comprise or consist of a 10, 11, 12, 13, 14, 15, 16, 17, or 18 nucleotide fragment of the sequence defined in SEQ ID NO: 8 or 30.

Alternatively, the first strand may comprise or consist of the sequence defined in SEQ ID NO: 9 or 31, or variants or fragments of the sequence defined in SEQ ID NO: 9 or 31. The variants may comprise or consist of a sequence having at least 80%, at least 85%, at least 90%, at least 95% or at least 97% sequence identity to the sequence defined in SEQ ID NO: 9 or 31. Where an overhang is specified, the variants may include different overhangs. The fragments may comprise or consist of a 10, 11, 12, 13, 14, 15, 16, 17, or 18 nucleotide fragment of the sequence defined in SEQ ID NO: 9 or 31. The second strand may comprise or consist of the sequence defined in SEQ ID NO: 10 or 32 or variants or fragments of the sequence defined in SEQ ID NO: 10 or 32. The variants may comprise or consist of a sequence having at least 80%, at least 85%, at least 90%, at least 95% or at least 97% sequence identity to the sequence defined in SEQ ID NO: 10 or 32. Where an overhang is specified, the variants may include different overhangs. The fragments may comprise or consist of a 10, 11, 12, 13, 14, 15, 16, 17, or 18 nucleotide fragment of the sequence defined in SEQ ID NO: 10 or 32.

Alternatively, the first strand may comprise or consist of the sequence defined in SEQ ID NO: 11 or 33, or variants or fragments of the sequence defined in SEQ ID NO: 11 or 33. The variants may comprise or consist of a sequence having at least 80%, at least 85%, at least 90%, at least 95% or at least 97% sequence identity to the sequence defined in SEQ ID NO: 11 or 33.

Where an overhang is specified, the variants may include different overhangs. The fragments may comprise or consist of a 10, 11, 12, 13, 14, 15, 16, 17, or 18 nucleotide fragment of the sequence defined in SEQ ID NO: 11 or 33. The second strand may comprise or consist of the sequence defined in SEQ ID NO: 12 or 34, or variants or fragments of the sequence defined in SEQ ID NO: 12 or 34. The variants may comprise or consist of a sequence having at least 80%, at least 85%, at least 90%, at least 95% or at least 97% sequence identity to the sequence defined in SEQ ID NO: 12 or 34. Where an overhang is specified, the variants may include different overhangs. The fragments may comprise or consist of a 10, 11, 12, 13, 14, 15, 16, 17, or 18 nucleotide fragment of the sequence defined in SEQ ID NO: 12 or 34.

Alternatively, the first strand may comprise or consist of the sequence defined in SEQ ID NO: 13 or 35, or variants or fragments of the sequence defined in SEQ ID NO: 13 or 35. The variants may comprise or consist of a sequence having at least 80%, at least 85%, at least 90%, at least 95% or at least 97% sequence identity to the sequence defined in SEQ ID NO: 13 or 35.

Where an overhang is specified, the variants may include different overhangs. The fragments may comprise or consist of a 10, 11, 12, 13, 14, 15, 16, 17, or 18 nucleotide fragment of the sequence defined in SEQ ID NO: 13 or 35. The second strand may comprise or consist of the sequence defined in SEQ ID NO: 14 or 36, or variants or fragments of the sequence defined in SEQ ID NO: 14 or 36. The variants may comprise or consist of a sequence having at least 80%, at least 85%, at least 90%, at least 95% or at least 97% sequence identity to the sequence defined in SEQ ID NO: 14 or 36. Where an overhang is specified, the variants may include different overhangs. The fragments may comprise or consist of a 10, 11, 12, 13, 14, 15, 16, 17, or 18 nucleotide fragment of the sequence defined in SEQ ID NO: 14 or 36.

Alternatively, the first strand may comprise or consist of the sequence defined in SEQ ID NO: 15 or 37, or variants or fragments of the sequence defined in SEQ ID NO: 15 or 37. The variants may comprise or consist of a sequence having at least 80%, at least 85%, at least 90%, at least 95% or at least 97% sequence identity to the sequence defined in SEQ ID NO: 15 or 37.

Where an overhang is specified, the variants may include different overhangs. The fragments may comprise or consist of a 10, 11, 12, 13, 14, 15, 16, 17, or 18 nucleotide fragment of the sequence defined in SEQ ID NO: 15 or 37. The second strand may comprise or consist of the sequence defined in SEQ ID NO: 16 or 38, or variants or fragments of the sequence defined in SEQ ID NO: 16 or 38. The variants may comprise or consist of a sequence having at least 80%, at least 85%, at least 90%, at least 95% or at least 97% sequence identity to the sequence defined in SEQ ID NO: 16 or 38. Where an overhang is specified, the variants may include different overhangs. The fragments may comprise or consist of a 10, 11, 12, 13, 14, 15, 16, 17, or 18 nucleotide fragment of the sequence defined in SEQ ID NO: 16 or 38.

Alternatively, the first strand may comprise or consist of the sequence defined in SEQ ID NO: 17 or 39, or variants or fragments of the sequence defined in SEQ ID NO: 17 or 39. The variants may comprise or consist of a sequence having at least 80%, at least 85%, at least 90%, at least 95% or at least 97% sequence identity to the sequence defined in SEQ ID NO: 17 or 39.

Where an overhang is specified, the variants may include different overhangs. The fragments may comprise or consist of a 10, 11, 12, 13, 14, 15, 16, 17, or 18 nucleotide fragment of the sequence defined in SEQ ID NO: 17 or 39. The second strand may comprise or consist of the sequence defined in SEQ ID NO: 18 or 40, or variants or fragments of the sequence defined in SEQ ID NO: 18 or 40. The variants may comprise or consist of a sequence having at least 80%, at least 85%, at least 90%, at least 95% or at least 97% sequence identity to the sequence defined in SEQ ID NO: 18 or 40. Where an overhang is specified, the variants may include different overhangs. The fragments may comprise or consist of a 10, 11, 12, 13, 14, 15, 16, 17, or 18 nucleotide fragment of the sequence defined in SEQ ID NO: 18 or 40.

Alternatively, the first strand may comprise or consist of the sequence defined in SEQ ID NO: 19 or 41, or variants or fragments of the sequence defined in SEQ ID NO: 19 or 41. The variants may comprise or consist of a sequence having at least 80%, at least 85%, at least 90%, at least 95% or at least 97% sequence identity to the sequence defined in SEQ ID NO: 19 or 41. Where an overhang is specified, the variants may include different overhangs. The fragments may comprise or consist of a 10, 11, 12, 13, 14, 15, 16, 17, or 18 nucleotide fragment of the sequence defined in SEQ ID NO: 19 or 41. The second strand may comprise or consist of the sequence defined in SEQ ID NO: 20 or 42, or variants or fragments of the sequence defined in SEQ ID NO: 20 or 42. The variants may comprise or consist of a sequence having at least 80%, at least 85%, at least 90%, at least 95% or at least 97% sequence identity to the sequence defined in SEQ ID NO: 20 or 42. Where an overhang is specified, the variants may include different overhangs. The fragments may comprise or consist of a 10, 11, 12, 13, 14, 15, 16, 17, or 18 nucleotide fragment of the sequence defined in SEQ ID NO: 20 or 42.

### Pharmaceutical Compositions, Dosages and Administration Routes

The inhibitory nucleic acids of the present invention can be incorporated into pharmaceutical compositions. These may be prepared using methods known to those of ordinary skill in the art. Non-limiting examples of suitable methods are described in Gennaro et al. (Eds), (1990), “Remington's Pharmaceutical Sciences”, Mack Publishing Co., Easton, Pennsylvania, USA.

The pharmaceutical compositions may comprise a pharmaceutically acceptable carrier, excipient, and/or diluent. “Pharmaceutically acceptable” carriers, excipients, and/or diluents as contemplated herein are substances which do not produce adverse reaction(s) when administered to a particular recipient such as a human or non-human animal. Pharmaceutically acceptable carriers, excipients, and diluents are generally also compatible with other ingredients of the composition. Non-limiting examples of suitable excipients, diluents, and carriers can be found in the “Handbook of Pharmaceutical Excipients” 4th Edition, (2003) Rowe et al. (Eds), The Pharmaceutical Press, London, American Pharmaceutical Association, Washington. Non-limiting examples of pharmaceutically acceptable carriers, excipients and diluents include demineralised or distilled water; saline solution; vegetable based oils such as peanut oil, safflower oil, olive oil, cottonseed oil, maize oil, sesame oils, arachis oil or coconut oil; silicone oils, including polysiloxanes, such as methyl polysiloxane, phenyl polysiloxane and methylphenyl polysiloxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

The pharmaceutical compositions can be provided in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, or in a form suitable for parenteral administration, that is, intradermal, subcutaneous, intramuscular or intravenous injection. Injectable solutions or suspensions may employ non-toxic parenterally acceptable diluents or carriers such as, for example, Ringer's solution, isotonic saline, phosphate buffered saline, ethanol and 1,2 propylene glycol.

The pharmaceutical compositions may include any suitable surfactant such as an anionic, cationic or non-ionic surfactant such as sorbitan esters or polyoxyethylene derivatives thereof.

Suspending agents such as natural gums, cellulose derivatives or inorganic materials such as silicaceous silicas, and other ingredients such as lanolin, may also be included.

In an embodiment, the inhibitory nucleic acids described herein are administered without a delivery system associated with the molecule either covalently or noncovalently i.e. as naked siRNA.

In an embodiment, the inhibitory nucleic acids described herein are the pharmaceutical composition are protected, for example by encapsulation or conjugation to a ligand. The pharmaceutical compositions may be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used.

The pharmaceutical compositions in liposome form may contain stabilisers, preservatives, excipients and the like. The preferred lipids are phospholipids and phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq.

The pharmaceutical compositions may be formulated as lipid nanoparticles. Any suitable liquid nanoparticle delivery system may be used, as known to those of ordinary skill in the art.

For example, the inhibitory nucleic acids described herein may be formulated with a lipid nanoparticle composition comprising a cationic lipid/Cholesterol/PEG-DMG/DSPC, for example, in a 40/48/2/10 ratio, or a cationic lipid/Cholesterol/PEG-C-DMA/DSPC, for example, in a 40/48/2/10 ratio. The cationic lipid may, for example, be CLinDMA or DLinDMA. The PEG may, for example, be PEG-DMG. Other suitable and non-limiting lipid nanoparticle delivery systems include those described in the Examples of the present application; Idris et al. 2021, “A SARS-CoV-2 targeted siRNA-nanoparticle therapy for COVID-19”, Molecular Therapy Vol. 29 No 7, 2219-2226; Wu et al. 2008, “Development of a Novel Method for Formulating Stable siRNA-Loaded Lipid Particlesfor In vivo Use”, Pharmaceutical Research, Vol. 26, No. 3, 512-522; and U.S. Pat. Nos. 7,514,099, 9,061,063, 10,369,226, 11,071,784, and 11,382,979).

The pharmaceutical compositions may be administered in the form of exosomes. As used herein the term “exosome” refers to a cell-derived small (between 20-300 nm in diameter, more preferably 40-200 nm in diameter) vesicle comprising a membrane that encloses an internal space, and which is generated from said cell by direct plasma membrane budding or by fusion of the late endosome with the plasma membrane. The exosome comprises lipid or fatty acid and polypeptide and further comprises the inhibitory nucleic acids described herein as a payload. The exosome can be derived from a producer cell, and isolated from the producer cell based on its size, density, biochemical parameters, or a combination thereof. Exosomes can be directly loaded with exogenous nucleic acids or drugs by electroporation, lipofection, sonication and contact with calcium chloride. Alternatively, purified exosomes may be loaded ex vivo by, for example, electroporation.

Exosomes of the present invention can be produced from a cell grown in vitro or a body fluid of a subject. When exosomes are produced from in vitro cell culture, various producer cells, e.g., HEK293 cells, Chinese hamster ovary (CHO) cells, or mesenchymal stem cells (MSCs), can be used.

The pharmaceutical compositions may also be formulated by incorporation of the inhibitory nucleic acids described herein into adenoviruses or adeno-associated viruses (AAVs), formulated with cell-penetrating peptides, lentiviral vectors, polymers, dendrimers, or prepared as siRNA bioconjugates such as the GalNAc-siRNA conjugate delivery platform.

If using the exosomes or a vector as a vehicle to deliver siRNA, the candidate siRNAs are delivered as shRNAs. Both siRNAs and shRNAs can target and repress viruses and are functionally equivalent. When the candidate siRNAs are delivered as shRNAs they are derived from a cell system and packaged into exosomes or a vector (AAV or Lentiviral vector) as described above.

An shRNA may be provided in an expression cassette containing a promoter contiguously linked to an siRNA as described herein. In embodiments, the promoter is a polII or a polIII promoter, such as a U6 promoter (e.g., a mouse U6 promoter) or a H1 promoter. In embodiments, the expression cassette further contains a marker gene. In embodiments, the promoter is a polII promoter. In embodiments, the promoter is a tissue-specific promoter. In embodiments, the promoter is an inducible promoter. In embodiments, the promoter is a polIII promoter. In embodiments, the promoter is U6 or H1 promoter.

Also provided is a vector containing an expression cassette described herein. Examples of appropriate vectors include adenoviral, lentiviral, adeno-associated viral (AAV), poliovirus, herpes simplex virus (HSV), or murine Maloney-based viral vectors. In an embodiment, the vector is an adeno-associated virus (AAV) vector.

An shRNA molecule comprises paired RNA sequences and a loop portion positioned between the paired RNA sequences so as to form the hairpin. The loop can vary in length. In some embodiments the loop is 5, 6, 7, 8, 9, 10, 11, 12, 13, 14, 15, 16, 17, 18, 19, 20, 21, 22, 23, 24 or 25 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length. The nucleotide sequence of the loop region may vary and could be, for example, (5′-GCAA-3′), (5′-GCGC-3′) or (5′-TTGC-3′) or other sequences as will be well understood by the skilled person.

The pharmaceutical compositions described herein may be administered in dosages sufficient to inhibit the expression of the target gene or the biological activity of non-translated target sequences (e.g. regulatory sequences) in a cell, tissue or organism under treatment. The specific dosages of the inhibitory nucleic acids described herein administered to a given subject will depend on factors such as the route of administration and physical characteristics of the subject (including health status) and so forth. For example, the appropriate dosage of a given pharmaceutical composition comprising the inhibitory nucleic acids described herein may depend on a variety of factors including, but not limited to, a subject's physical characteristics (e.g. age, weight, sex), the progression (i.e. pathological state) of a given coronavirus infection, and other factors that will be readily recognised by one skilled in the art. Various general considerations that may be considered when determining an appropriate dosage are described, for example, in Gennaro et al. (Eds), (1990), “Remington's Pharmaceutical Sciences”, Mack Publishing Co., Easton, Pennsylvania, USA; and Gilman et al. (Eds), (1990), “Goodman And Gilman's: The Pharmacological Bases of Therapeutics”, Pergamon Press. Non-limiting examples of suitable dosages of the inhibitory nucleic acids described herein include those in the range of 0.01 to 200 milligrams per kilogram body weight of the recipient per day, 1 to 50 mg/kg body weight per day, 1 to 40 mg/kg body weight per day, 1 to 30 mg/kg body weight per day, 1 to 20 mg/kg body weight per day, 1 to 10 mg/kg body weight per day, 1 to 5 mg/kg body weight per day, 1 to 3 mg/kg body weight per day, 1 to 2 mg/kg body weight per day, 0.1 to 1 mg/kg body weight per day, 0.1 to 0.9 mg/kg body weight per day, 0.1 to 0.8 mg/kg body weight per day, 0.1 to 0.7 mg/kg body weight per day, 0.1 to 0.6 mg/kg body weight per day, 0.1 to 0.5 mg/kg body weight per day, 0.1 to 0.4 mg/kg body weight per day, 0.1 to 0.3 mg/kg body weight per day, 0.1 to 0.2 mg/kg body weight per day, 0.01 to 0.1 mg/kg body weight per day, 0.01 to 0.05 mg/kg body weight per day, 0.01 to 0.02 mg/kg body weight per day, and 0.005 to 0.01 mg/kg body weight per day.

Those of ordinary skill in the art will be able, by routine experimentation, to determine an effective, non-toxic amount of the pharmaceutical compositions and/or inhibitory nucleic acids described herein to include in a dosage or in a series of dosages to achieve the desired therapeutic outcome.

Typically, in therapeutic applications, the treatment would be for the duration of the infection, disease state or condition. Further, it will be apparent to one of ordinary skill in the art that the optimal quantity and spacing of individual dosages will be determined by the nature and extent of the infection, disease state or condition being treated, the form, route and site of administration, and the nature of the particular individual being treated. Such optimum conditions can also be determined using conventional techniques.

In many instances, it will be desirable to have several or multiple administrations of a pharmaceutical composition described herein. For example, they may be administered 1, 2, 3, 4, 5, 6, 7, 8, 9, 10, or more times. The administrations may be from about one to about twelve week intervals, and in certain embodiments from about one to about four week intervals. Periodic re-administration may be desirable in the case of recurrent exposure to a particular pathogen targeted by a pharmaceutical composition described herein.

It will also be apparent to one of ordinary skill in the art that the optimal course of treatment can be ascertained using conventional course of treatment determination tests.

Suitable techniques for introduction of the inhibitory nucleic acids described herein into cells, tissues, and organisms include various carrier systems, vectors and reagents. Non-limiting examples include lipid nanoparticles (LNP), micelles, nucleic-acid-lipid particles, lipoplexes, liposomes, nucleic acid polymers, single chemical entity conjugates, virosomes, virus like particles (VLP), and mixtures thereof.

Pharmaceutical compositions of the present invention may be administered in any suitable way, such as, for example, intravenously, buccally, parenterally, intranasally, orally, sublingually, or topically. Accordingly, the administration may be topical, pulmonary (e.g. by inhalation or insufflation of aerosols or powders including with a nebulizer), intranasal, intratracheal, epidermal, transdermal, oral or parenteral. Parenteral administration includes intravenous, intraarterial, subcutaneous, intraperitoneal or intramuscular injection or infusion; or intracranial (e.g. intraparenchymal, intrathecal or intraventricular) administration.

In an embodiment, the pharmaceutical composition is adapted for intranasal administration. Significant antiviral targeting in both the lungs and nasal cavities of SARS-CoV-2 infected mice can be achieved when pharmaceutical compositions of the present invention are delivered intranasally.

In an embodiment, a pharmaceutical composition of the present invention is formulated as a direct-acting nasal spray. In an embodiment, a nasal spray can be self-administered at point-of-care.

### Therapeutic Methods

In general, the inhibitory nucleic acids described herein are demonstrated to inhibit, reduce or prevent the replication of coronaviruses.

Accordingly, various aspects of the present invention provide methods for inhibiting coronavirus replication, methods for treating coronavirus infection, and methods for treating conditions and diseases arising from coronavirus infection, in subjects in need thereof.

In some embodiments, there is provided a method for inhibiting replication of a coronavirus in a cell, the method comprising administering an inhibitory nucleic acid described herein, which may be included in a vector, lipid nanoparticle or pharmaceutical composition, to the cell to thereby cause degradation of a coronavirus mRNA molecule in the cell and inhibit replication of the coronavirus.

In other embodiments, there is provided a method for treating a coronavirus infection in a subject, the method comprising administering an inhibitory nucleic acid described herein, which may be included in a vector, lipid nanoparticle or pharmaceutical composition, to thereby inhibit replication of the coronavirus and treat the infection.

In other embodiments, there is provided a method for treating COVID-19 disease in a subject, the method comprising administering a therapeutically effective amount of an inhibitory nucleic acid described herein, which may be included in a vector, lipid nanoparticle or pharmaceutical composition, to the subject, to thereby inhibit replication of the coronavirus and treat said COVID-19 disease.

In still other embodiments, there is provided use of an inhibitory nucleic acid described herein, which may be included in a vector, lipid nanoparticle or pharmaceutical composition, in the preparation of a medicament for inhibiting replication of a coronavirus in a cell, treating a coronavirus infection in a subject, or treating COVID-19 disease in a subject.

In further embodiments, there is provided an inhibitory nucleic acid described herein, which may be included in a vector, lipid nanoparticle or pharmaceutical composition, for use in inhibiting replication of a coronavirus in a cell, treating a coronavirus infection in a subject, or treating COVID-19 disease in a subject.

In an embodiment, administration is intranasal. Significant antiviral targeting in both the lungs and nasal cavities of SARS-CoV-2 is achieved in infected mice when pharmaceutical compositions of the present invention are delivered intranasally. While not wishing to be bound by theory, it is believed that an intranasally-delivered anti-SARS-CoV-2 siRNA antiviral that can ameliorate viral replication in the nasal cavity and prevent aerosol spread of respiratory viruses.

In an embodiment an encapsulated siRNA is delivered intranasally.

In an embodiment, administration is by way of a direct-acting nasal spray. In an embodiment, a nasal spray can be self-administered at point-of-care.

For many respiratory viruses including SARS-CoV-2, the nasal cavity is a primary target for respiratory viral replication in the early stages of disease and a critical source of virus spread via aerosols. RNAi is cost effective, scalable and can be easily programmed to target any viral RNA. The approach could result in the ushering in of an entirely new cost-effective programmable RNA platform technology and self-administrable IN delivery platform, but also could deliver the first in class RNA drugs suitable for emergence of any new RNA respiratory viruses of concern.

The subject may be any animal of economic, social or research importance including bovine, equine, ovine, primate, avian and rodent species. Accordingly, the subject may be a mammal such as, for example, a human or a non-human mammal (e.g. a pig, cat, dog, cow, horse, or sheep). The subject may be a laboratory animal (e.g. a rodent such as a mouse, rat, or guinea pig; a rabbit, and the like), a bird (e.g. poultry), a fish or a crustacean.

The treatment methods may be used on subjects infected by alpha coronaviruses (e.g. HCoV-229E and HCoV-NL63), or beta coronaviruses (e.g. SARS-CoV-1, SARS-CoV-2, MERS-CoV, HCoV-HKU1 or HCoV-OC43.) The treatment methods may be used to treat subjects diagnosed with conditions or diseases arising from coronavirus infection, including, but not limited to, severe acute respiratory syndrome, coronavirus disease 2019 (COVID-19), and Middle East respiratory syndrome.

The identification of subjects in need of treatment by the methods described herein may comprise those identified by testing for coronavirus infection and/or those identified by assessment of symptoms.

Subjects positive for coronavirus infection can be identified using standard techniques known in the art including, for example, CT-scan, PCR-based methods (e.g. cycle threshold (CT) value, qRT-PCR), sequencing, CRISPR, ELISA, LFA, RT-LAMP, Colloidal gold immunolateral flow chromatography.

Symptoms of coronavirus infection are varied, and in many cases difficult to detect.

Additionally, overlap exists with symptoms arising from other independent sources. Accordingly, in some embodiments a subject may be assessed for symptoms consistent with coronavirus infection, followed by a diagnostic test to confirm or refute that the symptoms arising from a coronavirus infection. Non-limiting examples of symptoms arising from coronavirus infection include, but are not limited to, fever, cough, fatigue, loss of taste or smell, sore throat, headache, muscle or joint pains, nausea or loss of appetite, diarrhoea, vomiting, difficulty breathing or shortness of breath, loss of speech or mobility, confusion, and/or chest pain.

The efficacy of the treatment methods described herein can be assessed using standard methods known to those of ordinary skill in the art. For example, PCR-based techniques (e.g. qRT-PCR) for assessing coronavirus load during or following therapy may be used to assess the effectiveness of the methods of treatment described herein. General techniques capable of assessing the efficacy of the treatment methods described herein include in vitro plaque assays, and the use of in vivo mouse models. Additionally or alternatively, techniques employed for the diagnosis of coronavirus infection including CRISPR, ELISA, LFA, RT-LAMP, Colloidal gold immunolateral flow chromatography can be used to assess therapeutic efficacy, particularly if adapted to provide quantitative data.

### EXAMPLE(S)

The present invention will now be described with reference to specific Example(s), which should not be construed as in any way limiting.

### Example One: siRNA Targeting of Sars-Cov

### Materials and Methods

(a) siRNA Design

Several candidate siRNAs were developed using an algorithm (https://weinbergmorrislab.wixsite.com/weinbergmorrislab/tgs-algorithm-format; see also Ackley et al. 2013, “An Algorithm for Generating Small RNAs Capable of Epigenetically Modulating Transcriptional Gene Silencing and Activation in Human Cells”, Molecular Therapy-Nucleic Acids (2013) 2, e104) that selectively finds siRNA seed sequences based on purine tracts in the target RNA. Regions of the viral genome that are ultra-conserved and highly susceptible to siRNA targeting and contain the 4-6 bp purine tract were selected. These non-chemically modified siRNAs were designed against various conserved sites in the SARS-CoV-2 genome including the 5′ untranslated region (5′UTR) conserved stem, the helicase region, and a conserved region in the RNA-dependent RNA polymerase (RdRp) gene.

### (b) Interferon Screening

THP-1 DUAL cells, a well-recognized standard to measure immunostimulation, were transfected with indicated siRNAs using Fugene 6 for 24 h before quantifying for (A) IRF and (B) NFkB gene reporter expression levels. 2′3′-cGAMP (20 g/ml) and LPS (100 ng/ml) were used as positive controls for IRF and NFkB pathway stimulation, respectively.

### (c) Plaque Assays

The ability of the various siRNAs to inhibit SARS-CoV-2 (Vic-1; Wuhan) was assessed using plaque assays. VeroE6 cells were either untreated (virus), pre-treated without (Lipo+virus) or with 30 nM of target siRNA complexed with Lipofectamine 2000 for 24 h before infection with Wuhan (ancestral) SARS-CoV-2 at 250PFU. Top new candidate siRNAs were also infected with Alpha, Beta, Kappa, Delta or Omicron variant SARS-CoV-2 at 250PFU. Post treatment, viral plaques were counted after 4 days. Data was collected from triplicate treatments.

(d) qRT-PCR Screening

VeroE6 cells were either pre-treated without (Lipo alone) or with siRNA complexed with Lipofectamine 2000 for 24 hours before infection with SARS-CoV-2 at 250PFU. Combinations of the siRNAs were selected, mixed in equal molar ratios to a final concentration of 30 nM and viral copy numbers were determined by digital droplet PCR against the N gene at 4 dpi. Data is representative of the range of the mean of duplicate treatments.

### (e) Dose Response Evaluation for Delta Variant Repression

Viral genes and phylogenetically conserved regions, targeting candidate siRNAs were designed and selected for screening against SARS-CoV-2 infected cells. VeroE6 cells were either pre-treated without (Lipo+virus) or with seven dilutions (30, 20, 10, 5, 2.5, 1 or 0.1 nM) of target siRNA complexed with Lipofectamine 2000 for 24 h before infection with Delta variant SARS-CoV-2 at 250PFU. Post treatment, viral plaques were counted after 4 days. Data was collected from triplicate treatments.

(f) Liponanoparticle (LNP) Delivery of siRNAs

K18hACE2 mice infected with 5×10⁴ live delta SARS-CoV-2 variant received prophylactic intravenous (retro-orbital) treatment of siRNA-LNP (1 mg/kg) daily (−1 to 2 dpi). Lung viral tissue counts/g at 3 dpi are shown. Each dot represents data from one mouse. The siRNA Hel2 found previously to repress SARS-CoV-2 was contrasted with the new siRNA siHELUp2.

### Results

There are several methods for preparing siRNA, such as chemical synthesis, in vitro transcription, siRNA expression vectors, and PCR expression cassettes. In chemical synthesis, commercial suppliers provide siRNAs with dTdT 3′ overhangs. As an alternative, the overhangs can be UJU or other overhangs. The skilled person will appreciate that there is no substantial difference in the potency of siRNA made with dTdT or UJU overhangs and that the composition of the overhang does not appear to play a critical role in target mRNA recognition and cleavage. Overhangs are not part of the target site in the mRNA but rather useful for other purposes e.g. Ago-2 and slicer activity and loading into the complex.

The candidate siRNAs developed are shown in Table 1. The siRNAs in Table 1 have dTdT overhangs and are loaded into an LNP in this form, but could equally have UJU overhangs or another overhang such as an overhang complementary to the mRNA sequence, or the overhang could be absent.

TABLE 1
siRNAs targeted to various loci in the SARS-CoV-2 virus. The siRNAs include dTdT
overhangs. The top performing siRNAs are highlighted in bold/underline.

| siRNA | RNA sequence sense (5′-3′) | RNA sequence antisense (5′-3′) |
|---|---|---|
| SL5 si1 | AGAUGGAGAGCCUUGUCCCtt | GGGACAAGGCUCUCCAUCUtt |
| (siCoV 9) | (SEQ ID NO: 1) | (SEQ ID NO: 2) |
| siHELUp1 | CGUGGUAAGAGAAUUCCUUtt | AAGGAAUUCUCUUACCACGtt |
| (siCoV 4) | (SEQ ID NO: 3) | (SEQ ID NO: 4) |
| siHELUp2 | GAGAAAAGCUGUCUUUAUUtt | AAUAAAGACAGCUUUUCUCtt |
| (siCoV 1) | (SEQ ID NO: 5) | (SEQ ID NO: 6) |
| siUC7TGS- | CUUACUAAAGGACCUCAUGtt | CAUGAGGUCCUUUAGUAAGtt |
| 1 | (SEQ ID NO: 7) | (SEQ ID NO: 8) |
| (siCoV_10) | | |
| siUC7_TGS- | AUUAUCAAAACAAUGUUUUtt | AAAACAUUGUUUUGAUAAUtt |
| 2 | (SEQ ID NO: 9) | (SEQ ID NO: 10) |
| (siCoV_8) | | |
| siUC7_TGS- | AUGUCUGAAGCAAAAUGUUtt | AACAUUUUGCUUCAGACAUtt |
| 3 | (SEQ ID NO: 11) | (SEQ ID NO: 12) |
| (siCoV_7) | | |
| siHEL_TGS- | UUUUGGGACUACCAACUCAtt | UGAGUUGGUAGUCCCAAAAtt |
| 1 | (SEQ ID NO: 13) | (SEQ ID NO: 14) |
| (siCoV_6) | | |
| siHEL_TGS- | CCUCAAAGAUUUUGGGACUtt | AGUCCCAAAAUCUUUGAGGtt |
| 2 | (SEQ ID NO: 15) | (SEQ ID NO: 16) |
| (siCoV_5) | | |
| siHELdwn1 | UGACUAUGUCAUAUUCACUtt | AGUGAAUAUGACAUAGUCAtt |
| (siCoV_3) | (SEQ ID NO: 17) | (SEQ ID NO: 18) |
| siHELdwn2 | ACCACUGAAACAGCUCACUtt | AGUGAGCUGUUUCAGUGGUtt |
| (siCoV_2) | (SEQ ID NO: 19) | (SEQ ID NO: 20) |
| N367 | CUGACCUUUGGAUGGUGCUtt | AGCACCAUCCAAAGGUCAGtt |
| (control) | (SEQ ID NO: 21) | (SEQ ID NO: 22) |

TABLE 2
Target site for the siRNAs of Table 1. The dTdT overhang is not shown.

| siRNA | RNA sequence sense (5′-3′) | RNA sequence antisense (5′-3′) |
|---|---|---|
| SL5_si1 | AGAUGGAGAGCCUUGUCCC | GGGACAAGGCUCUCCAUCU |
| (siCoV_9) | (SEQ ID NO: 23) | (SEQ ID NO: 24) |
| siHELUp1 | CGUGGUAAGAGAAUUCCUU | AAGGAAUUCUCUUACCACG |
| (siCoV_4) | (SEQ ID NO: 25) | (SEQ ID NO: 26) |
| siHELUp2 | GAGAAAAGCUGUCUUUAUU | AAUAAAGACAGCUUUUCUC |
| (siCoV_1) | (SEQ ID NO: 27) | (SEQ ID NO: 28) |
| siUC7TGS-1 | CUUACUAAAGGACCUCAUG | CAUGAGGUCCUUUAGUAAG |
| (siCoV_10) | (SEQ ID NO: 29) | (SEQ ID NO: 30) |
| siUC7_TGS-2 | AUUAUCAAAACAAUGUUUU | AAAACAUUGUUUUGAUAAU |
| (siCoV_8) | (SEQ ID NO: 31) | (SEQ ID NO: 32) |
| siUC7_TGS-3 | AUGUCUGAAGCAAAAUGUU | AACAUUUUGCUUCAGACAU |
| (siCoV_7) | (SEQ ID NO: 33) | (SEQ ID NO: 34) |
| siHEL_TGS-1 | UUUUGGGACUACCAACUCA | UGAGUUGGUAGUCCCAAAA |
| (siCoV_6) | (SEQ ID NO: 35) | (SEQ ID NO: 36) |
| siHEL_TGS-2 | CCUCAAAGAUUUUGGGACU | AGUCCCAAAAUCUUUGAGG |
| (siCoV_5) | (SEQ ID NO: 37) | (SEQ ID NO: 38) |
| siHELdwn1 | UGACUAUGUCAUAUUCACU | AGUGAAUAUGACAUAGUCA |
| (siCoV_3) | (SEQ ID NO: 39) | (SEQ ID NO: 40) |
| siHELdwn2 | ACCACUGAAACAGCUCACU | AGUGAGCUGUUUCAGUGGU |
| (siCoV_2) | (SEQ ID NO: 41) | (SEQ ID NO: 42) |
| N367 | CUGACCUUUGGAUGGUGCU | AGCACCAUCCAAAGGUCAG |
| (control) | (SEQ ID NO: 43) | (SEQ ID NO: 44) |

Sequences referenced for the purposes of comparison are shown in Tables 3 and 4.

TABLE 3
siRNAs prepared by Idris et al (2021) and included for comparison. The siRNAs
include dTdT overhangs.

| siRNA | RNA sequence sense (5′-3′) | RNA sequence antisense (5′-3′) |
|---|---|---|
| UC7_as1 | GACAUAAAAACAUUGUUUUtt | AAAACAAUGUUUUUAUGUCtt |
| (siCoV_1as) | (SEQ ID NO: 45) | (SEQ ID NO: 46) |
| UC7_as2 | AAACAUUGUUUUGAUAAUAtt | UAUUAUCAAAACAAUGUUUtt |
| (siCoV_2as) | (SEQ ID NO: 47) | (SEQ ID NO: 48) |
| UC7_as3 | GUAUGUUGAGAGCAAAAUUtt | AAUUUUGCUCUCAACAUACtt |
| (siCoV_3as) | (SEQ ID NO: 49) | (SEQ ID NO: 50) |
| UC7_as4 | UAAUCAUCACCCUGUUUAAtt | UUAAACAGGGUGAUGAUUAtt |
| (siCoV_4as) | (SEQ ID NO: 51) | (SEQ ID NO: 52) |
| UC7_as5 | AUUUUGCUUCAGACAUAAAtt | UUUAUGUCUGAAGCAAAAUtt |
| (siCoV_5as) | (SEQ ID NO: 53) | (SEQ ID NO: 54) |
| UTR5_as | CUACAAGAGAUCGAAAGUUtt | AACUUUCGAUCUCUUGUAGtt |
| (siCoV_6as) | (SEQ ID NO: 55) | (SEQ ID NO: 56) |
| Hel4_as | CUGUUUCAGUGGUUUGAGUtt | ACUCAAACCACUGAAACAGtt |
| (siCoV_7as) | (SEQ ID NO: 57) | (SEQ ID NO: 58) |
| Hel5_as | GGUUUGAGUGAAUAUGACAtt | UGUCAUAUUCACUCAAACCtt |
| (siCoV_8as) | (SEQ ID NO: 59) | (SEQ ID NO: 60) |

TABLE 4
Target sites in the siRNAs prepared by Idris et al (2021) and included for
comparison. The dTdT overhang is not shown.

| SIRNA | RNA sequence sense (5′-3′) | RNA sequence antisense (5′-3′) |
|---|---|---|
| UC7_as1 | GACAUAAAAACAUUGUUUU | AAAACAAUGUUUUUAUGUC |
| (siCoV_1as) | (SEQ ID NO: 61) | (SEQ ID NO: 62) |
| UC7_as2 | AAACAUUGUUUUGAUAAUA | UAUUAUCAAAACAAUGUUU |
| (siCoV_2as) | (SEQ ID NO: 63) | (SEQ ID NO: 64) |
| UC7_as3 | GUAUGUUGAGAGCAAAAUU | AAUUUUGCUCUCAACAUAC |
| (siCoV_3as) | (SEQ ID NO: 65) | (SEQ ID NO: 66) |
| UC7_as4 | UAAUCAUCACCCUGUUUAA | UUAAACAGGGUGAUGAUUA |
| (siCoV_4as) | (SEQ ID NO: 67) | (SEQ ID NO: 68) |
| UC7_as5 | AUUUUGCUUCAGACAUAAA | UUUAUGUCUGAAGCAAAAU |
| (siCoV_5as) | (SEQ ID NO: 69) | (SEQ ID NO: 70) |
| UTR5_as | CUACAAGAGAUCGAAAGUU | AACUUUCGAUCUCUUGUAG |
| (siCoV_6as) | (SEQ ID NO: 71) | (SEQ ID NO: 72) |
| Hel4_as | CUGUUUCAGUGGUUUGAGU | ACUCAAACCACUGAAACAG |
| (siCoV_7as) | (SEQ ID NO: 73) | (SEQ ID NO: 74) |
| Hel5_as | GGUUUGAGUGAAUAUGACA | UGUCAUAUUCACUCAAACC |
| (siCoV_8as) | (SEQ ID NO: 75) | (SEQ ID NO: 76) |

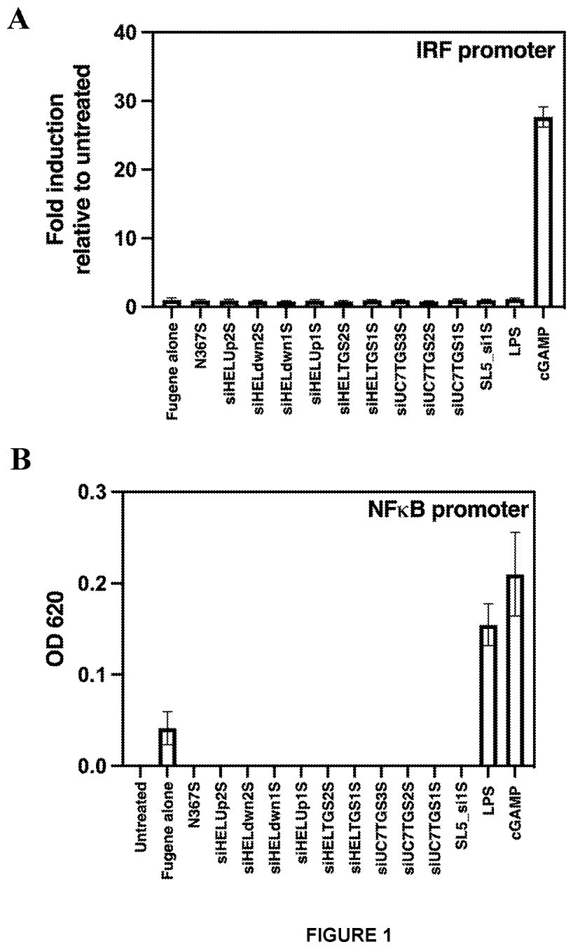

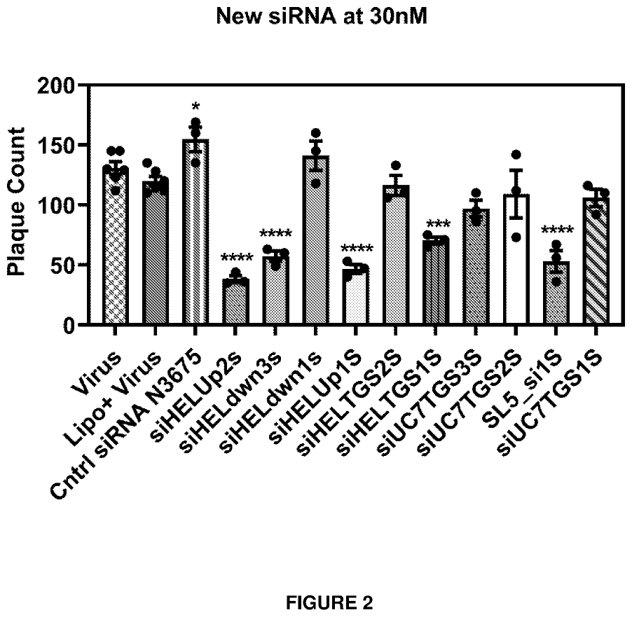

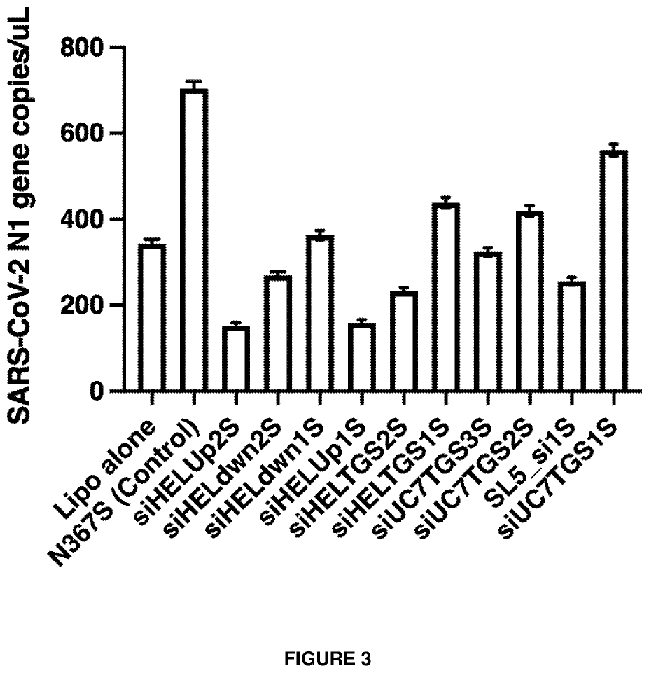

Regions of the viral genome that are ultra-conserved and highly susceptible to siRNA targeting and contain the 4-6 bp purine tract were selected and screened for any interferon activation (FIGS. 1A-B). None of the siRNAs induced any notable interferon activation, suggesting that they are non-immunogenic. Next, to determine the ability of the various siRNAs to inhibit SARS-CoV-2, Vero cells were transfected and then infected with the SARS-CoV-2. Several candidates appeared to robustly repress SARS-CoV-2, including siHELUp2ps, siHELdwn3s, SL5-si1S as determined by plaque assay (FIG. 2) and qRT-PCR (FIG. 3).

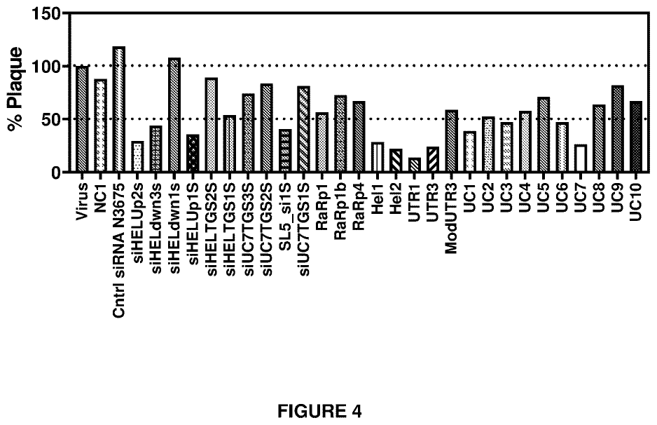

Next, a contrast of the effectiveness of these new algorithm candidate siRNAs was contrasted with previously published siRNAs found to potently repress the Wuhan SARS-CoV-2 variant (see Idris et al. 2021, Ibid) (FIG. 4). The top candidate siRNAs appeared to be conserved to particular regions where a previous study also found potent siRNA candidates including the Helicase locus, e.g. siHel2, RNA-dependent RNA polymerase, e.g. siUC7, and the 5′ UTR, e.g. siUTR3 (see Idris et al. 2021, Ibid) (FIG. 4).

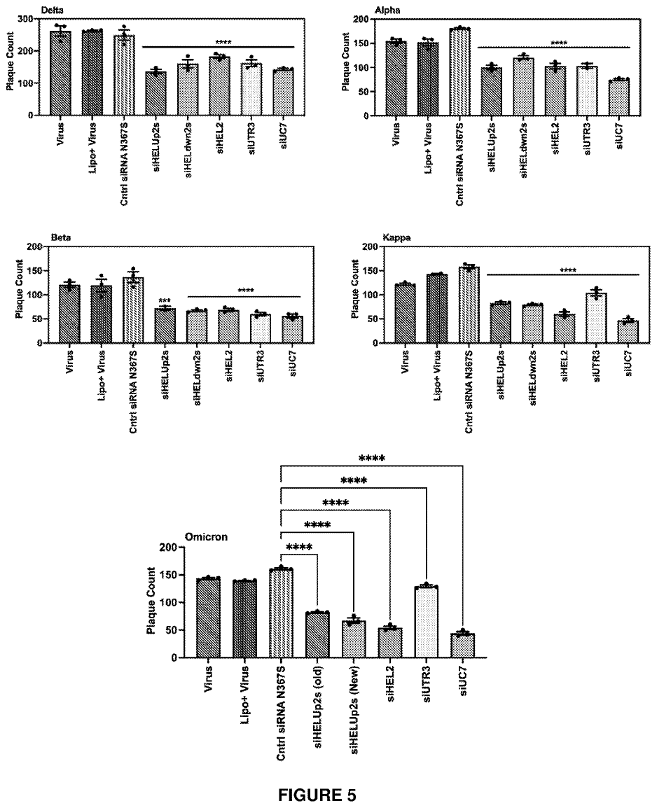

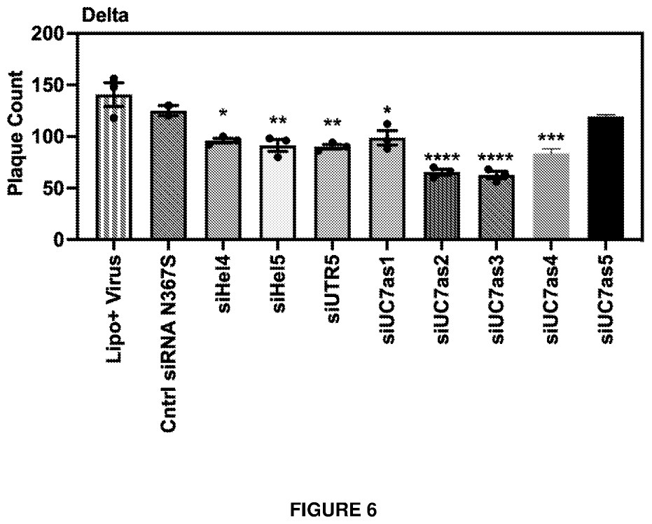

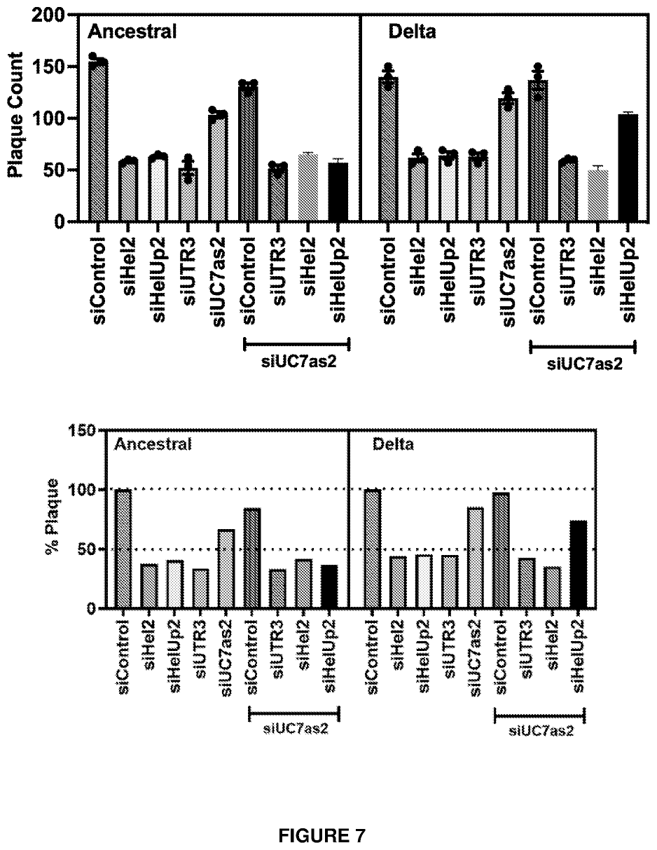

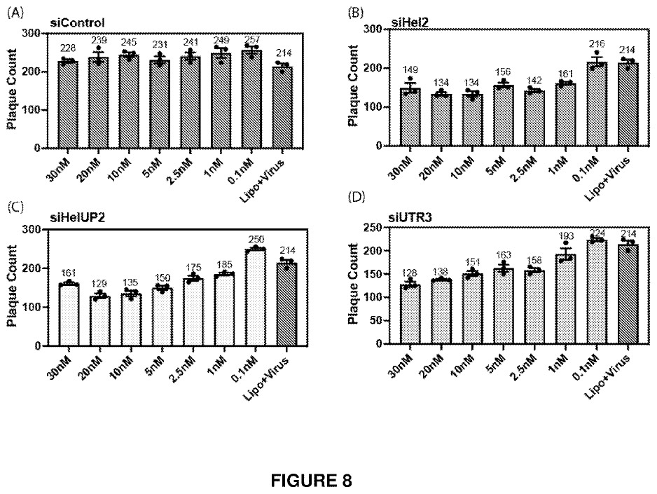

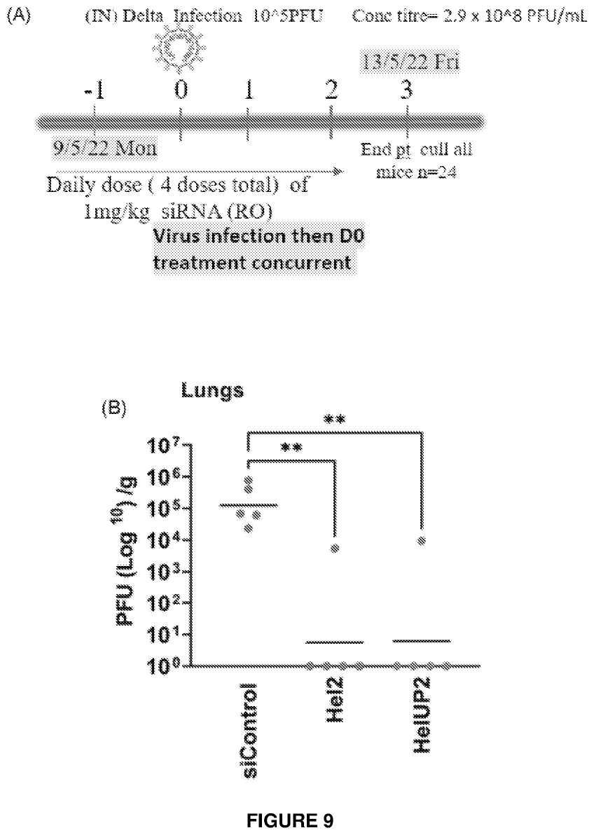

There are several variants of SARS-CoV-2, including alpha, beta, kappa, and delta. The top candidate siRNAs, including those published previously were screened for their effectiveness against the various viral variants. All of the siRNAs tested, including the algorithm generated siRNAs were found to be effective at repressing the various viral variants (FIG. 5). The SARS-CoV-2 virus undergoes negative RNA strand synthesis in the viral lifecycle. The effectiveness of siRNAs targeted to this step in the viral life cycle was interrogated and it was observed that some repression of viral expression was instilled following the negative strand targeting of the delta variant (FIG. 6). To determine the abilities of combinations of siRNAs, sense strand and negative strand targeting siRNAs to repress SARS-CoV-2 and if there is an added benefit to such combinations, single and dual treated cells challenged with virus were contrasted and it was observed that combinations of siRNAs do not greatly enhance or diminish the observed repression (FIG. 7). Next, a dose response of the top three candidate siRNAs was carried out for repression of the delta variant in vitro (FIG. 8). The siRNA target sites of the top three siRNAs in the new omicron variant of SARS-CoV-2 were also assessed and it was observed that all siRNAs contain 100% sequence targeting homology to the new omicron variant. Lastly, the top candidate siRNA, siHELUp2, was selected and contrasted with the previously reported siHEL2 (see Idris et al. 2021, Ibid) for repression of the delta SARS-CoV-2 virus in vivo. Notably, siHELUp2 repressed SARS-CoV-2 as well as siHel2 in vivo (FIG. 9), suggesting that siHELUp2 is also a good candidate siRNA for broadscale repression of SARS-CoV-2.

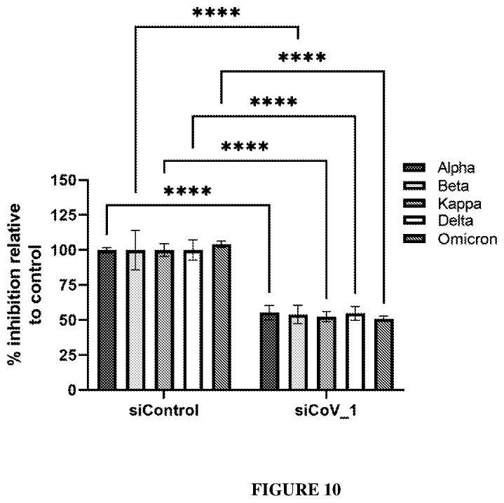

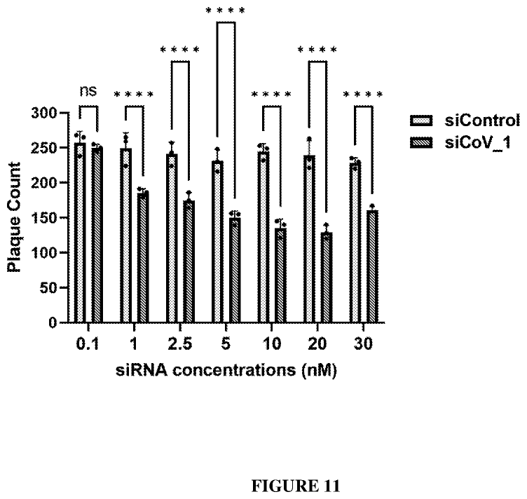

siHELUp2 (siCoV_1), which targets a conserved site in the helicase gene region of SARS-CoV-2, was then screened for its effectiveness against the various SARS-CoV-2 VOCs and was found to be equipotent across all tested VOCs including the omicron variant (FIG. 10). Upon bioinformatically assessing the siRNA target site of siHELUp2 in the omicron variant we confirm that this siRNA exhibited 100% sequence targeting homology to this variant further underscoring the ultra-conserved design of this siRNA. A dose response of siHELUp2 against SARS-CoV-2 revealed an IC₅₀ of 3.99 nM compared to siControl (IC₅₀=1.3×10¹² nM) (FIG. 11). SARS-CoV-2 undergoes negative RNA strand synthesis in its viral lifecycle.

Collectively these data highlight that the algorithm defined siRNAs are functional in repressing all the variants of SARS-CoV-2 and that the top three candidate siRNAs all function equally well and can be used in LNP based delivery approaches as a therapeutic for COVID-19.

### Example Two: Intranasal Administration

### Materials and Methods

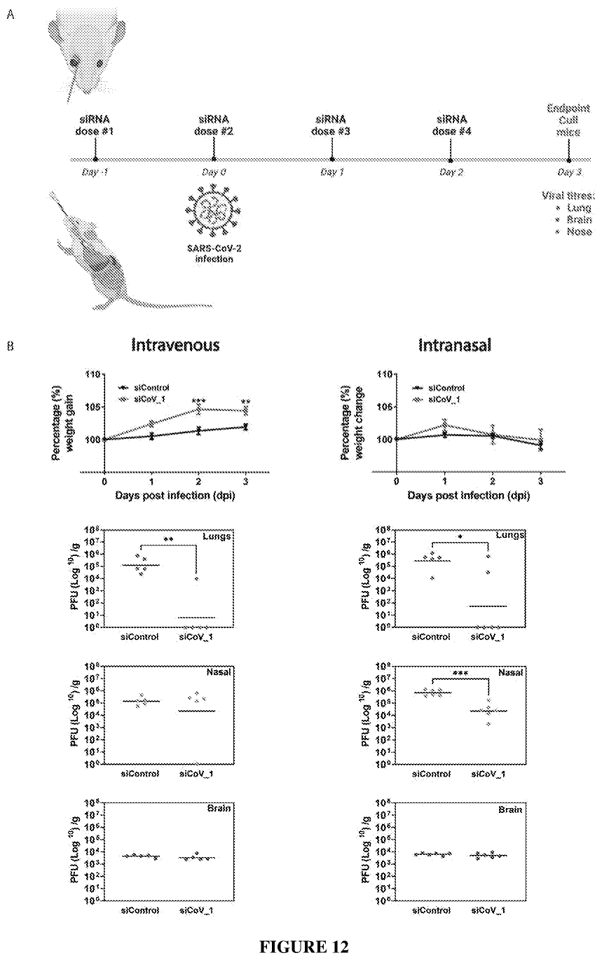

Mice were intranasally (IN) infected using the IN instillation technique with 10⁵ plaque forming unit (PFU) (20 L total volume) of live delta SARS-CoV-2 VOC while under isoflurane anesthesia. Mice were subsequently treated with sLNP or dmLNP complexed siRNAs retro-orbitally (IV) (100 L total volume) or IN (20 L total volume) administered while under isoflurane anesthesia. For siRNA dosing on the day of infection (0 days post-infection (dpi)), siRNA was administered 2 h prior to infecting mice with SARS-CoV-2. Mice were monitored daily for weighing and clinical scoring. We employed the same prophylactic treatment strategy as done previously in SARS-CoV-2 infected K18-hACE2 mice using LNP-based delivery of siRNAs (Supramaniam et al.) (FIG. 12A).

### Results

It was observed that IV-delivered siHELUp2 (siCoV_1) repressed SARS-CoV-2 in the lungs of infected mice when formulated in ‘stealth LNP’ (sLNP) (FIG. 12B). We then formulated siHELUp2 in dmLNPs, a lung-targeting LNP shown to biodistribute and retain siRNAs in the nasal cavity (Supramaniam et al.). Remarkably, IN delivered siHELUp2-LNP significantly reduces not only lung viral loads but also nasal viral loads. This is the first demonstration of an anti-SARS-CoV-2 siRNA targeting both the lower and upper respiratory tract. Collectively these data highlight that the algorithm defined siRNA, siHELUp2, is functional in repressing all the variants of SARS-CoV-2 and that it can be used in LNP-based delivery to reduce viral burden in the lungs and nasal cavities of mice.

### Example 3—shRNAs

If using exosomes or a vector as a vehicle to deliver siRNA, the candidate siRNAs are delivered as shRNAs. Both siRNAs and shRNAs can target and repress viruses and are functionally equivalent. When the candidate siRNAs are delivered as shRNAs they are derived from a cell system and packaged into exosomes or a vector (AAV or Lentiviral vector). They do not have the dtdT overhangs, but rather just the sequences which are shown in Table 2.

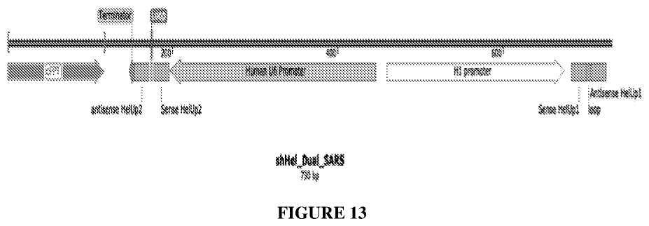

An shRNA is provided in an expression cassette containing a promoter contiguously linked to an siRNA. A dual-shRNA expression cassette (FIG. 13) may be used to express siHELUp2 and siHELUp1 for packaging into EVs. Both H1 and U6 promoters are shown in FIG. 13, but each promoter and shRNA can be expressed alone, or as in the case here, in combination. The nucleotide sequence of the loop region is (5′-GCAA-3′) but could also be (5′-GCGC-3′) or (5′-TTGC-3′) or other sequences as will be well understood by the skilled person.

The sequence of the insert shown in FIG. 13 is as follows:

(SEQ ID NO: 77)
5′-gaaaaaaGAGAAAAGCTGTCTTTATTGCAAAATAAAGACAGCTTTT
CTCcggatcttcgtcctttccacaagatatataaagccaagaaatcgaa
atactttcaagttacggtaagcatatgatagtccattttaaaacataat
tttaaaactgcaaactacccaagaaattattactttctacgtcacgtat
tttgtactaatatctttgtgtttacagtcaaattaattccaattatctc
tctaacagccttgtatcgtatatgcaaatatgaaggaatcatgggaaat
aggccctcactagtatcaattcgaacgctgacgtcatcaacccgctcca
aggaatcgcgggcccagtgtcactaggcgggaacacccagcgcgcgtgc
gccctggcaggaagatggctgtgagggacaggggagtggcgccctgcaa
tatttgcatgtcgctatgtgttctgggaaatcaccataaacgtgaaatg
tctttggatttgggaatcttataagttctgtatgagaccacagatctag
CGTGGTAAGAGAATTCCTTttgcAAGGAATTCTCTTACCACGttttt
t-3′

### REFERENCES

The content of the following documents is incorporated herein by reference:
Wang H, Paulson K R, Pease S A, Watson S, Comfort H, Zheng P, et al. Estimating excess mortality due to the COVID-19 pandemic: a systematic analysis of COVID-19-related mortality, 2020-21. The Lancet. 2022; 399(10334):1513-36.
Meganck R M, Baric R S. Developing therapeutic approaches for twenty-first-century emerging infectious viral diseases. Nature Medicine. 2021; 27(3):401-10.
Cao Z, Gao W, Bao H, Feng H, Mei S, Chen P, et al. VV116 versus Nirmatrelvir-Ritonavir for Oral Treatment of Covid-19. New England Journal of Medicine. 2022; 388(5):406-17.
Fischer W, Eron J J, Holman W, Cohen M S, Fang L, Szewczyk L J, et al. Molnupiravir, an Oral Antiviral Treatment for COVID-19. medRxiv. 2021.
Takashita E, Kinoshita N, Yamayoshi S, Sakai-Tagawa Y, Fujisaki S, Ito M, et al. Efficacy of Antibodies and Antiviral Drugs against Covid-19 Omicron Variant. N Engl J Med. 2022; 386(10):995-8.
Idris A, Davis A, Supramaniam A, Acharya D, Kelly G, Tayyar Y, et al. A SARS-CoV-2 targeted siRNA-nanoparticle therapy for COVID-19. Mol Ther. Vol. 29 No 7, 2219-2226. 2021.
Zhang X, Goel V, Robbie G J. Pharmacokinetics of Patisiran, the First Approved RNA Interference Therapy in Patients With Hereditary Transthyretin-Mediated Amyloidosis. The Journal of Clinical Pharmacology. 2020; 60(5):573-85.
To K K, Tsang O T, Leung W S, Tam A R, Wu T C, Lung D C, et al. Temporal profiles of viral load in posterior oropharyngeal saliva samples and serum antibody responses during infection by SARS-CoV-2: an observational cohort study. Lancet Infect Dis. 2020; 20(5):565-74.
Chang Y-C, Yang C-F, Chen Y-F, Yang C-C, Chou Y-L, Chou H-W, et al. A siRNA targets and inhibits a broad range of SARS-CoV-2 infections including Delta variant. EMBO Molecular Medicine. 2022; 14(4):e15298.
Supramaniam A, Tayyar Y, Clarke D T W, Kelly G, Acharya D, Morris K V, et al. Prophylactic intranasal administration of lipid nanoparticle formulated siRNAs reduce SARS-CoV-2 and RSV lung infection. Journal of Microbiology, Immunology and Infection. 2023; 56(3):516-25.
Amarilla A A, Modhiran N, Setoh Y X, Peng N Y G, Sng J D J, Liang B, et al. An Optimized High-Throughput Immuno-Plaque Assay for SARS-CoV-2. Front Microbiol. 2021; 12:625136.
Khaitov M, Nikonova A, Shilovskiy I, Kozhikhova K, Kofiadi I, Vishnyakova L, et al. Silencing of SARS-CoV-2 with modified siRNA-peptide dendrimer formulation. Allergy. 2021; 76(9):2840-54.
Bowden-Reid E, Ledger S, Zhang Y, Di Giallonardo F, Aggarwal A, Stella A O, et al. Novel siRNA therapeutics demonstrate multi-variant efficacy against SARS-CoV-2. Antiviral Research. 2023; 217:105677.
Ackley A, Lenox A, Stapleton K, Knowling S, Lu T, Sabir K S, et al. An Algorithm for Generating Small RNAs Capable of Epigenetically Modulating Transcriptional Gene Silencing and Activation in Human Cells. Molecular Therapy—Nucleic Acids. 2013; 2(7):e104.
McCaskill J L, Marsh G A, Monaghan P, Wang L F, Doran T, McMillan N A. Potent inhibition of Hendra virus infection via RNA interference and poly I:C immune activation. PLoS One. 2013; 8(5):e64360.
Brutlag et al. Improved sensitivity of biological sequence database searches. Computer Applications in the Biosciences. 1990; 6:237-245.
Usman et al. The automated chemical synthesis of long oligoribuncleotides using 2′-O-silylated ribonucleoside 3′-O-phosphoramidites on a controlled-pore glass support: synthesis of a 43-nucleotide sequence similar to the 3′-half molecule of an Escherichia coli formylmethionine tRNA. Journal of the American Chemical Society. 1987; 109(25):7845-54.
Scaringe et al. Chemical synthesis of biologically active oligoribonucleotides using beta-cyanoethyl protected ribonucleoside phosphoramidites. Nucleic Acids Research. 1990; 18(18):5433-41.
Bellon et al. Amino-linked ribozymes: post-synthetic conjugation of half-ribozymes. Nucleosides & Nucleotides. 1997; 16(7-9):951-54.
Bellon et al. Post-synthetically ligated ribozymes: an alternative approach to iterative solid-phase synthesis. Bioconjugate Chemistry. 1997; 8(2):204-12.
Shabarova et al. Chemical ligation of DNA: the first non-enzymatic assembly of a biologically active gene. Nucleic Acids Research. 1991; 19(15):4247-51.
Moore et al. Site-specific modification of pre-mRNA: the 2′-hydroxyl groups at the splice sites. Science. 1992; 256(5059):992-7.
Wu et al. Development of a Novel Method for Formulating Stable siRNA-Loaded Lipid Particles for In vivo Use. Pharmaceutical Research, 2008; 26(3):512-522.

1. An isolated nucleic acid or an isolated fragment thereof, comprising 15 to 30 nucleotides and capable of specifically hybridising to a SARS-CoV-2 sequence as defined in SEQ ID NO: 23, SEQ ID NO: 25, or SEQ ID NO: 27, or capable of hybridising to a fragment of said SARS-CoV-2 sequence.

2. The isolated nucleic acid or the isolated fragment thereof of claim 1, comprising or consisting of: 15 to 25 nucleotides; 15 to 23 nucleotides; 15 to 22 nucleotides; 15 to 21 nucleotides; 15 to 20 nucleotides; 15 to 19 nucleotides; 19 nucleotides; 20 nucleotides; 21 nucleotides, 22 nucleotides or 23 nucleotides.

3. An isolated nucleic acid having at least 80%, at least 85%, at least 90%, at least 95% or at least 97% sequence identity to:
a sequence as defined in SEQ ID NO: 2 or 24, wherein the nucleic acid is capable of specifically hybridising to a SARS-CoV-2 sequence as defined in SEQ ID NO: 23; or
a sequence as defined in SEQ ID NO: 4 or 26, wherein the nucleic acid is capable of specifically hybridising to a SARS-CoV-2 sequence as defined in SEQ ID NO: 25; or
a sequence as defined in SEQ ID NO: 6 or 28, wherein the nucleic acid is capable of specifically hybridising to a SARS-CoV-2 sequence as defined in SEQ ID NO: 27.

4. The isolated nucleic acid or the isolated fragment thereof claim 1, wherein the isolated fragment is 10, 11, 12, 13, 14, 15, 16, 17, or 18 nucleotides in length.

5. An isolated double stranded nucleic acid for inhibiting expression of SARS-CoV-2, comprising a sense strand and an antisense strand, wherein:
the sense strand comprises a sequence as defined in SEQ ID NO: 1 or 23 or a variant or fragment thereof, and the antisense strand comprises a sequence as defined in SEQ ID NO: 2 or 24 or a variant or fragment thereof, or
the sense strand comprises a sequence as defined in SEQ ID NO: 3 or 25 or a variant or fragment thereof, and the antisense strand comprises a sequence as defined in SEQ ID NO: 4 or 26 or a variant or fragment thereof, or
the sense strand comprises a sequence as defined in SEQ ID NO: 5 or 27 or a variant or fragment thereof, and the antisense strand comprises a sequence as defined in SEQ ID NO: 6 or 28 or a variant or fragment thereof.

6. The isolated nucleic acid or the isolated fragment thereof of claim 1, comprising: a 2′-deoxy-2′-fluoro modified nucleotide; a 2′-deoxy-modified nucleotide; a locked nucleotide; an abasic nucleotide; 2′-amino-modified nucleotide; 2′-alkyl-modified nucleotide; morpholino nucleotide; a non-natural base comprising nucleotide; a 2′-O-methyl modified nucleotide; a 2′-O-methoxyethoxy modified nucleotide; a 2′-fluoro modified nucleotide; a 5-methyl-modified cytidine; pseudouridine; a nucleotide comprising a 5′-phosphorothioate group and a terminal nucleotide linked to a cholesteryl derivative or dodecanoic acid bisdecylamide group; a nucleotide comprising phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite; or a nucleotide comprising deoxyribose.

7. The isolated nucleic acid or the isolated fragment thereof of claim 1, which is conjugated to a ligand.

8. A cell comprising the isolated nucleic acid or the isolated fragment thereof of claim 1.

9. The isolated nucleic acid or the isolated fragment thereof of claim 1, which is RNA, antisense RNA, or siRNA.

10. A vector comprising a nucleotide sequence that encodes the RNA, antisense RNA, or siRNA of claim 9.

11. A lipid nanoparticle comprising
the isolated nucleic acid or the isolated fragment thereof of claim 1.

12. The lipid nanoparticle of claim 11, wherein the lipid comprises any one or more of: a non-cationic lipid, a cationic lipid, a conjugated lipid for preventing aggregation of the nanoparticle.

13. A pharmaceutical composition comprising
the isolated nucleic acid or the isolated fragment thereof of claim 1
and a pharmaceutically acceptable excipient, carrier or diluent.

14. The pharmaceutical composition of claim 13, further comprising a liposome, AAV or exosome, wherein the isolated nucleic acid or the isolated fragment thereof is protected.

15. (canceled)

16. (canceled)

17. The pharmaceutical composition of claim 13, comprising naked siRNA or an shRNA.

18. (canceled)

19. The pharmaceutical composition of claim 13, which is a liquid for intravenous administration or an aerosol for intranasal administration.

20. (canceled)

21. A method for inhibiting replication of a coronavirus in a cell, the method comprising administering the isolated nucleic acid or the isolated fragment thereof of claim 1, to the cell to thereby cause degradation of a coronavirus mRNA molecule in the cell and inhibit said replication of the coronavirus.

22. A method for treating a coronavirus infection in a subject, the method comprising administering a therapeutically effective amount of the nucleic acid or the isolated fragment thereof of claim 1 to the subject to thereby inhibit replication of the coronavirus and treat said infection.

23. A method for treating COVID-19 disease in a subject, the method comprising administering a therapeutically effective amount of the isolated nucleic acid or the isolated fragment thereof of claim 1 to the subject to thereby inhibit replication of the coronavirus and treat said COVID-19 disease.

24. (canceled)

25. (canceled)

26. The method of claim 23, wherein the coronavirus is SARS-CoV-2.
